SEQUENCE LISTING (33 sequences)

SEQ ID NO: 1 - DNA, 1000 bp, Hevea brasiliensis
cgggttaagtcagtgatttaaggaaaatggaattatacaacggtgagaggccaagtgtgttcagacttttagagaagtatatgagaaaagggttatatagcatcctaacccagggtcccatccctactcatattgccttcatattggatggaaacaggaggtttgctaagaagcataaactgccagaaggaggtggtcataaggctggatttttagctcttctgaacgtactaacttattgctatgagttaggagtgaaatatgcgactatctatgcctttagcatcgataattttcgaaggaaacctcatgaggttcagtacgtaatggatctaatgctggagaagattgaagggatgatcatggaagaaagtatcatcaatgcatatgatatttgcgtacgttttgtgggtaacctgaagcttttaagtgagccagtcaagaccgcagcagataagattatgagggctactgccaacaattccaaatgtgtgcttctcattgctgtatgctatacttcaactgatgagatcgtgcatgctgttgaagaatcctctgaattgaactccaatgaagtttgtaacaatcaagaattggaggaggcaaatgcaactggaagcagtactgtgattcaaactgagaacatggagtcgtattctggaataaaacttgtagaccttgagaaaaacacctacataaatccttatcctgatgttctgattcgaacttctggggagacccgtctgagcaactacttactttggcagactactaattgcatactgtattctccttatgcactgtggccagagattggtcttcgacacgtggtgtggtcagtaattaacttccaacgtcattattcttacttggagaaacataaggaatacttaaaataatttgtttctgttcctagctcatcctgccttattcgcgatagttaagcttaagcatatccttgtggaataaactcggacacttaattaagccggtattttgt

SEQ ID NO: 2 - protein, 290 aa, Hevea brasiliensis
MELYNGERPSVFRLLEKYMRKGLYSILTQGPIPTHIAFILDGNRRFAKKHKLPEGGGHKAGFLALLNVLTYCYELGVKYATIYAFSIDNFRRKPHEVQYVMDLMLEKIEGMIMEESIINAYDICVRFVGNLKLLSEPVKTAADKIMRATANNSKCVLLIAVCYTSTDEIVHAVEESSELNSNEVCNNQELEEANATGSSTVIQTENMESYSGIKLVDLEKNTYINPYPDVLIRTSGETRLSNYLLWQTTNCILYSPYALWPEIGLRHVVWSVINFQRHYSYLEKHKEYLK

SEQ ID NO: 3 - DNA, 900 bp, Oryza sativa
atgcttggctcacttatgtcttacttaccttcagtggattcaaagacggagaacactgatgagttaattgcgactggtgttcttgctagtctgcagaatttcatccgcaaatgcattgtagctgtcctctcgtatggcccaatgcctaagcatattgcatttattatggatggtaaccgtagatatgctaaattcaggagtatccaggaaggctctggtcacagggtgggcttctctgctctcattgccagcctgctctactgctatgaaatgggcgtgaagtatatcacggtgtatgcatttagcatcgataattttaagcgagatccgactgaggtgaaatccttgatggagttaatggaggaaaagatcaatgaactgctagaaaacagaaatgtcatcaacaaggttaactgtaagatcaacttctgggggaacttggacatgttgagcaaatcagtgagggtagcagctgagaaactgatggctaccactgctgaaaacacgggactggtcttctctgtttgcatgccatacaactccacttctgagattgtcaatgcggtcaataaggtctgtgcagaaaggagggatatactgcagagggaggatgctgacagtgttgcgaataatggtgtgtattcagacatttcagtggcagatctggaccgccatatgtacagcgctggttgccccgatcctgacattgtgatccggacctcaggtgagactcgcctgagcaatttccttctgtggcagacgacgttcagtcatttgcagaatccagaccctctttggccggagttctctttcaagcaccttgtctgggccatactccagtaccaaagagttcacccttctattgagcaaagcagaaatctggctaagaagcagctgtaa

SEQ ID NO: 4 - protein, 299 aa, Oryza sativa
MLGSLMSYLPSVDSKTENTDELIATGVLASLQNFIRKCIVAVLSYGPMPKHIAFIMDGNRRYAKFRSIQEGSGHRVGFSALIASLLYCYEMGVKYITVYAFSIDNFKRDPTEVKSLMELMEEKINELLENRNVINKVNCKINFWGNLDMLSKSVRVAAEKLMATTAENTGLVFSVCMPYNSTSEIVNAVNKVCAERRDILQREDADSVANNGVYSDISVADLDRHMYSAGCPDPDIVIRTSGETRLSNFLLWQTTFSHLQNPDPLWPEFSFKHLVWAILQYQRVHPSIEQSRNLAKKQL

SEQ ID NO: 5 - DNA, 1028 bp, Glycine max
ttcccactcagtggtgaatttgccaaaccgggataaccgtatccctattcaggaatacaatgttctcgttaagactccctattcctctcgttaaaacaccaccttctccctcttgttattattctcactattatcactatcgttatcgttatcgttgttatcatcctttccatcaccgttcccaaacacagagtcttatcgtctcgaagcgcggttccgccattgcgaagtgtcacgctgatagcgtgacacttcgtgatgacggagtctcgctcgcccaagagtcgttggagccacttccggcggaactcgcggcggagatgatgccgaagcatgtggcggtgataatggacgggaacgggaggtgggcgaaggtgaaggggctgccaccatcggcggggcaccaggcgggggtgcaatcgctgaggaaaatggtgaggctgtgttgcagctggggaattaaggttctaacggttttcgcgttctctacggataactgggttcgccccaaggtggaggttgatttcttgatgaggctgtttgagagaacaataaactctgaagttcaaacttttaagagggaaggaattagaatatctgtgattggagattcatcaaggttgcctgagtctttaaaaagaatgatagctagtgcagaagaggatacaaaacaaaattcgagattccaacttattgtggcagtgggatacagtggaaaatatgatgttgtgcaagcatgtaaaagtgtagccaagaaagtcaaagatggtcacattcacttggatgacataaacgaaaacattattgaacaagaattggaaactaattgtactgagtttccttatcctgatctactaatacgaactagtggcgagcttagagtgagtaacttcttgttgtggcaattagcctacacagaactttattttaatcgggaactctggccagattttgggaaggatgagtttgtagatgcattaagttcatttcaacaaagacaaagacgctatggtggtcgacattcataa

SEQ ID NO: 6 - protein, 322 aa, Glycine max
MFSLRLPIPLVKTPPSPSCYYSHYYHYRYRYRCYHPFHHRSQTQSLIVSKRGSAIAKCHADSVTLRDDGVSLAQESLEPLPAELAAEMMPKHVAVIMDGNGRWAKVKGLPPSAGHQAGVQSLRKMVRLCCSWGIKVLTVFAFSTDNWVRPKVEVDFLMRLFERTINSEVQTFKREGIRISVIGDSSRLPESLKRMIASAEEDTKQNSRFQLIVAVGYSGKYDVVQACKSVAKKVKDGHIHLDDINENIIEQELETNCTEFPYPDLLIRTSGELRVSNFLLWQLAYTELYFNRELWPDFGKDEFVDALSSFQQRQRRYGGRHS

SEQ ID NO: 7 - protein, 13 aa, Artificial Sequence
  Consensus Sequence (Domain I) from Apfel CM et al. (J Bacteriol 1999 Jan;181(2)483-492)
HXXXXMDGNXRXA

SEQ ID NO: 8 - protein, 5 aa, Artificial Sequence
  Consensus Sequence (Domain II) from Apfel CM et al. (J Bacteriol 1999 Jan;181(2)483-492)
GHXXG

SEQ ID NO: 9 - protein, 23 aa, Artificial Sequence
  Consensus Sequence (Domain III) from Apfel CM et al. (J Bacteriol 1999 Jan;181(2)483-492)
XXXAFSXENXXRXXXEVXXLMXL

SEQ ID NO: 10 - protein, 35 aa, Artificial Sequence
  Consensus Sequence (Domain V) from Apfel CM et al. (J Bacteriol 1999 Jan;181(2)483-492)
XLXIRTXGEXRXSNFXXWQXXYXEXXFXXXXWPXF

SEQ ID NO: 11 - protein, 11 aa, Artificial Sequence
  Consensus Sequence (Domain I)
AFIXDGNRRFA

SEQ ID NO: 12 - protein, 10 aa, Artificial Sequence
  Consensus Sequence (Domain IV)
YXSXXXIXXA

SEQ ID NO: 13 - protein, 22 aa, Artificial Sequence
  Consensus Sequence (Domain V)
PXPDXLXRXSGXXRLSNXLLWQ

SEQ ID NO: 14 - DNA, 29 bp, Artificial Sequence
  Primer
gctctagaggttaagtcagtgatttaagg

SEQ ID NO: 15 - DNA, 36 bp, Artificial Sequence
  Primer
ggggtaccttattttaagtattccttatgtttctcc

SEQ ID NO: 16 - DNA, 26 bp, Artificial Sequence
  Primer
gtggatccatgcttggctcacttatg

SEQ ID NO: 17 - DNA, 26 bp, Artificial Sequence
  Primer
ttgagctctatctcctcccagggagg

SEQ ID NO: 18 - DNA, 27 bp, Artificial Sequence
  Primer
acggatccatgttctcgttaagactcc

SEQ ID NO: 19 - DNA, 26 bp, Artificial Sequence
  Primer
tcgagctcttatgaatgtcgaccacc

SEQ ID NO: 20 - DNA, 1029 bp, Arabidopsis thaliana
aacaaagtgtgttcttaaattatcttctctgataaccaaaaaagccctattttccgagatgaataccctagaagaagtagatgaatccactcatatcttcaacgctttgatgagtctaatgaggaaatttttgttcagagttctatgcgtcggtccaatccctactaacatttcattcatcatggatggaaaccgcaggttcgctaagaaacacaatcttataggcctagatgcaggacatagagctggtttcatatccgtgaaatatattcttcaatactgcaaagagattggtgtaccgtacgtcacactccacgcgtttggtatggataatttcaagagaggacctgaagaagtcaagtgtgtgatggatctaatgcttgagaaagtcgagctcgcgatcgatcaagctgtatcagggaatatgaacggcgtgagaataatctttgccggtgatttggattcgttaaacgagcattttagagctgcgacaaagaaactgatggagcttacggaggagaatagagatctgattgtggtggtttgcgttgcttacagcacaagtctcgagattgttcacgctgttcgaaaatcttgtgttagaaaatgtacgaatggagatgatcttgtacttttggagttgagtgatgttgaagagtgtatgtatacatcgattgtgccggttccggatcttgtgataagaaccggaggaggagatcggctgagtaacttcatgacgtggcaaacttcgaggtctcttcttcacagaacggaggctctttggccggagttagggctctggcatttggtttgggcaattcttaaattccaaagaatgcaagattacttgacgaagaagaaaaagctcgattagatagtttctaaagttaaaccctgcaggaaagaacttttaactctttattacgtttaatttacgtgtttctatgactggaaacgagaaagctcacaagcaaatcttttttattatgtattggatccgtataacaaacacgaatatacaaaacatcg

SEQ ID NO: 21 - protein, 271 aa, Arabidopsis thaliana
MNTLEEVDESTHIFNALMSLMRKFLFRVLCVGPIPTNISFIMDGNRRFAKKHNLIGLDAGHRAGFISVKYILQYCKEIGVPYVTLHAFGMDNFKRGPEEVKCVMDLMLEKVELAIDQAVSGNMNGVRIIFAGDLDSLNEHFRAATKKLMELTEENRDLIVVVCVAYSTSLEIVHAVRKSCVRKCTNGDDLVLLELSDVEECMYTSIVPVPDLVIRTGGGDRLSNFMTWQTSRSLLHRTEALWPELGLWHLVWAILKFQRMQDYLTKKKKLD

SEQ ID NO: 22 - DNA, 37 bp, Artificial Sequence
  Primer
ctagtctagaatctcccctccgataaccaaaaaatcc

SEQ ID NO: 23 - DNA, 34 bp, Artificial Sequence
  Primer
ggggtacctagggtttaacttagaaactatttag

SEQ ID NO: 24 - DNA, 24 bp, Artificial Sequence
  Primer
cgagatgaataccctagaagaagt

SEQ ID NO: 25 - DNA, 24 bp, Artificial Sequence
  Primer
gaaactatctaatcgagctttttc

SEQ ID NO: 26 - DNA, 24 bp, Artificial Sequence
  Primer
atggaattatacaacggtgagagg

SEQ ID NO: 27 - DNA, 24 bp, Artificial Sequence
  Primer
ttaagtattccttatgtttctcca

SEQ ID NO: 28 - DNA, 26 bp, Artificial Sequence
  Primer
gtggatccatgcttggctcacttatg

SEQ ID NO: 29 - DNA, 26 bp, Artificial Sequence
  Primer
ttgagctctatctcctcccagggagg

SEQ ID NO: 30 - DNA, 27 bp, Artificial Sequence
  Primer
acggatccatgttctcgttaagactcc

SEQ ID NO: 31 - DNA, 26 bp, Artificial Sequence
  Primer
tcgagctcttatgaatgtcgaccacc

SEQ ID NO: 32 - protein, 303 aa, Arabidopsis thaliana
MLSLLSSDSSLLSLLFLFLIPCLFITSYIGFPVFLLKLIGLIKIKAARDNEKRDEGTYVVREDGLQRELMPRHVAFILDGNRRWAKRAGLTTSQGHEAGAKRLIDIAELCFELGVHTVSAFAFSTENWGRDKIEIDNLMSLIQHYRNKSNIKFFHRSEVRVSVIGNKTKIPESLLKEIHEIEEATKGYKNKHLIMAVDYSGKFDIMHACKSLVKKSEKGLIREEDVDEALIERELLTNCSDFPSPDLMIRTSGEQRISNFFLWQLAYSELFFSPVFWPDFDKDKLLEALASYQRRERRFGCRV

SEQ ID NO: 33 - protein, 10 aa, Artificial Sequence
  Synthesized non-naturally occurring peptide
ELVISLIVES

(19) United States
(12) Patent Application Publication
(10) Pub. No.: US 2009/0288226 A1
(43) Pub. Date: Nov. 19, 2009

(54) Method for manipulating growth, yield, and architecture in plants

(76) Inventors: David L. Hallahan, Wilmington, DE (US); Natalie M. Keiper-Hrynko, Phoenixville, PA (US)

Correspondence Address: PIONEER HI-BRED INTERNATIONAL, INC., 7250 N.W. 62ND AVENUE, P.O. BOX 552, JOHNSTON, IA, 50131-0552, US

(21) Appl. No.: 10/557,504

(22) Filed: Nov. 21, 2005

(51) Int. Cl. C12N 15/82; A01H 5/00

(52) U.S. Cl. 800/287; 800/290; 800/298

(57) ABSTRACT

This invention pertains to a method for manipulating the growth rate and/or yield and/or architecture of a genetically modified plant, as compared to a corresponding wild-type plant. The method relies on over-expression of an endogenous or exogenous gene encoding a cis-prenyltransferase enzyme. In a preferred embodiment, transgenic plants obtained by this method exhibit increased growth rates to maturity, increased seed production, and increased height, siliques, and leave area.

This application claims the benefit of U.S. Provisional Application No. 60/472,813, filed May 22, 2003, which is hereby incorporated by reference in its entirety.

### FIELD OF THE INVENTION

This invention is in the field of plant molecular biology. More specifically, this invention provides a method of manipulating the growth rate, yield, and architecture of a genetically modified plant, as compared to a corresponding wild-type plant. The method relies on expression of cis-prenyltransferase genes in the plant host.

### BACKGROUND OF THE INVENTION

Plants exhibit growth rates dependent on the interplay of their genetics (genotypes) and the environmental conditions to which they are exposed during development. Under most conditions, the maximum growth potential of a plant is not realized. However, it has been demonstrated that manipulation of plant growth rate can be achieved by modification of the plant's genetic makeup, i.e., cultivated plant species may be bred to exhibit enhanced growth rates.

Genetic engineering of plants involves the isolation and manipulation of genetic material (e.g., DNA or RNA), followed by introduction of that genetic material into plants or plant cells. This means of “genetic enhancement” provides a methodology for rapid and directed alteration of the genetic makeup of cultivated plants, and this methodology represents an advance over traditional breeding methods that rely on selection of varieties or cultivars possessing and exhibiting the desired traits. In recent years, many beneficial traits have been introduced into plants using genetic engineering techniques (which often involve introduction of exogenous genes into the plant host), including: increased yield, pest and pathogen resistance, herbicide resistance, stress (e.g., drought) tolerance, etc.

One trait of particular interest to agricultural science is that of accelerated or increased growth rate to maturity. Such a trait is desirable, since plants would be capable of maturing in shorter growing seasons. This ability would permit expansion of the geographic range of cultivated species into climatic zones with shorter growing seasons, to thereby allow increased seed sales for the producer, bring benefit in the areas where the plants were newly adapted, and enable additional economic advantage to producers and consumers.

Accelerated growth to maturity of cultivated plants would be a distinct advantage in numerous applications. For example:
1. In silviculture, growth rate is a key economic property due to the generally slow-growing nature of trees. Applications of growth-influencing chemicals, currently extensively used, could be reduced or eliminated if genetically enhanced trees were available that exhibited properties of increased growth.
2. In agriculture, a focus of traditional breeding is expansion of the range of domesticated plants into cooler climatic zones with shorter growing seasons, with respect to the climatic zone in which the plant originated. Cereals originating in the Middle East (i.e., wheat) and Central America (i.e., corn) are now cultivated worldwide in different climatic zones. Numerous other plant species could be modified to exhibit accelerated growth to maturity, thus confering advantages to the world's human population.
3. In cell culture, where plants are generated for the production of valuable metabolites (e.g., drugs, drug precursors, vaccines, food additives), rapid cell growth is desirable to rapidly achieve high titers of the desired products.
4. In processes of plant genetic transformation, reduction in the overall time required for plant growth would be a significant improvement.
   Of course, the value of a trait for accelerated or increased growth rate would be significantly reduced if accompanied by deleterious effects in the plants so modified (e.g., reduction in seed number/fertility, negative alterations in the aesthetic or other commercially important aspects of the plant).

Previous work has recognized the need for a trait that confers accelerated or increased growth to maturity in plants. For example, U.S. Pat. No. 6,252,139 describes a method of producing a genetically enhanced plant with increased root growth and yield as a consequence of increased expression of genes encoding cyclin proteins. The transgenic plants exhibited increased main and lateral root growth rates, but effects on other tissues of the transgenic plants were not described.

WO 98/04725 describes a method for modulating the rate of plant development by modulating the amount of DNA methylation; in particular, an increase in methylated DNA (mediated by cytosine methyl transferase gene products) was found to correlate with an increased rate of growth to maturity.

WO 00/56905 describes a method for modifying plant growth, yield or architecture by increased expression of at least two cell cycle proteins. Specifically, unexpected alterations to plant architecture are described, as a consequence of overexpression of a protein kinase and a cyclin which interact together as a complex (e.g., plants overexpressing both genes exhibited increases in both root and shoot growth of between 10% and 30%). The results appeared to be based on an increase in cell number arising from an increased rate of cell division.

Finally, WO 01/66777 describes improvements in the growth rate and biomass of transgenic hybrid aspen trees following overexpression of a gibberellic acid 20-oxidase gene involved in gibberellin biosynthesis. Gibberellins are plant hormones well known for their effects on plant growth.

Although each of these above methods serves to modify plant growth, the art would be advanced by the identification of a novel gene sequence that conferred accelerated growth to maturity and increased yield to a genetically engineered plant, as compared to a corresponding wild-type plant. It would be particularly desirable to be able to manipulate the growth rate, and/or the yield, and/or the architecture of an entire plant, or specific target organs thereof.

Cis-prenyltransferase genes, which are known to catalyze the sequential addition of C₅ units to polyprenols and rubbers in cis 1-4 orientation, have not been previously recognized as capable of modifying plant phenotype and functioning to modify the growth rate to maturity, and/or yield, and/or architecture of a transformed plant, as compared to a corresponding wild-type plant.

### SUMMARY OF THE INVENTION

The invention provides a method for producing a transformed plant having an altered growth phenotype as compared with an untransformed plant comprising:
a) transforming a plant cell with a an isolated nucleic acid molecule encoding a cis-prenyltransferase under the control of suitable regulatory sequences;
b) recovering a transformed plant cell produced in step (a);
c) regenerating a plant from the transformed plant cell of step (b); and
d) growing the transformed plant produced in step (c) under conditions wherein the isolated nucleic acid molecule encoding a cis-prenyltransferase is expressed and the growth phenotype of the transformed plant is altered.

Preferred cis-prenyltransferase of the invention are those that comprise the domains as identified in SEQ ID NOs: 7-10 and SEQ ID NOs: 11-13.

In particular the expression of cis-prenyltransferase genes in plants has been demonstrated to affect plant growth rate, which may result in, decreased time to germination, increased root growth rate, increased shoot growth rate, decreased time to flowering, decreased time for fruit maturation, and decreased time of seed setting; increased yield as defined by increased total biomass, increased root growth, increased shoot growth, increased seed set, increased seed production, increased grain yield, increased fruit size, increased nitrogen fixing capacity, increased nodule size, increased tuber formation, increased stem thickness, increased endosperm size, and an increased number of fruit per plant; and modified plant architectural traits as defined by modifications in the shape, size, number, color, texture, arrangement and patternation of the root, leaf, shoot, fruit, petiole, trichome, flower, sepals, petal, hypocotyl, stigma, style, stamen, pollen, ovule, seed, embryo, endosperm, seed coat, aleurone, fibre nodule, cambium, wood, heartwood, parenchyma, sclerenchyma, seive element, phloem, or vascular tissue.

In an alternative embodiment the invention provides a method for altering the growth phenotype of a plant as compared with an untransformed plant comprising:
a) providing a plant comprising a gene encoding a cis-prenyltransferase; and
b) upregulating the gene of (a) wherein the growth phenotype of the plant is altered.
Plants, produced by the methods of the invention are also provided.

### BRIEF DESCRIPTION OF THE DRAWINGS AND SEQUENCE DESCRIPTIONS

FIG. 1 shows an alignment of the deduced amino acid sequences of the Hevea, rice, soybean and Arabidopsis Apt5 genes with that of Arabidopsis Apt1.

FIG. 2 is a gel showing the results of a reverse-transcriptase PCR of Arabidopsis lines transgenic for plant cis-prenyltransferase genes.

FIG. 3 visually compares the growth of a transgenic Arabidopsis expressing a Hpt2 cis-prenyltransferase and a wild-type plant, each 35 days after sowing, and grown under identical conditions.

FIG. 4 visually compares the growth of transgenic Arabidopsis lines expressing a Spt1 cis-prenyltransferase and a wild-type plant, each 35 days after sowing, and grown under identical conditions.

FIG. 5 visually compares the growth of a transgenic Arabidopsis expressing a Rpt1 cis-prenyltransferase and a wild-type plant, each 35 days after sowing, and grown under identical conditions.

FIG. 6 is a comparison between transgenic Arabidopsis expressing cis-prenyltransferases (Spt1, Rpt1, Hpt2, and Apt5, respectively) and wild-type plants, grown under identical conditions.

FIG. 7 visually compares the growth of a transgenic Arabidopsis plant expressing the cis-prenyltransferase Hpt2 and a wild-type plant, each 18 days after sowing, and grown under identical conditions.

FIG. 8 visually compares the growth of transgenic Arabidopsis plants expressing cis-prenyltransferases and a wild-type plant, each 28 days after sowing, and grown under identical conditions.

The invention can be more fully understood from the following detailed description and the accompanying sequence descriptions, which form a part of this application.

The following sequences comply with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. § 1.822.

SEQ ID NOs: 1-6, 20, 21 and 32 are genes or proteins as identified in Table 1.

TABLE 1
Summary of Gene and Protein SEQ ID Numbers

| | Clone ID | | SEQ ID | |
|---|---|---|---|---|
| Gene | number and | | Nucleic | SEQ ID |
| Name | Description | Organism | acid | Peptide |
| Hpt2 | ehb2c.pk001.d17 | Hevea | 1 | 2 |
| | | brasiliensis | | |
| Rpt1 | rr1.pk0050.h8 | Oryza sativa (rice) | 3 | 4 |
| Spt1 | sl1.pk0128.h7 | Glycine max | 5 | 6 |
| | | (soybean) | | |
| Apt5 | — | Arabidopsis | 20 | 21 |
| | | thaliana | | |
| | | (GenBank | | |
| | | AB011483) | | |
| Apt1 | — | Arabidopsis | | 32 |
| | | thaliana | | |

SEQ ID NOs:7-10 are consensus sequences representing conserved Domains I, II, III and V, as described by Apfel et al. (J. Bact. 182(2):483-492 (1999)).

SEQ ID NOs: 11-13 are consensus sequences representing modified conserved Domains I, IV, and V, that are indicative of the subfamily of cis-prenyltransferases associated with rubber-producing plants. These were described by Hallahan and Keiper-Hrynko in PCT/US03/36164.

SEQ ID NOs:14-19 are the primers HW8, HW12, JK1, JK2, JK3, and JK4, respectively.

SEQ ID NOs:22-25 are the primers Apt5/XbaI, Apt5/KpnI, Apt5s, Apt5 as, respectively.

SEQ ID NOs:26-31 are the primers H2s, H2 as, NHK33, NHK34, NHK35, and NHK36, respectively.

SEQ ID NO:33 is the peptide ‘ELVISLIVES’

### DETAILED DESCRIPTION OF THE INVENTION

The present invention provides a method for producing a genetically modified plant characterized as having a modified growth phenotype (as compared to a plant of the same species not genetically modified (i.e., a ‘wild-type’ plant)), by elevating the expression of cis-prenyltransferase gene(s) in the plant. The method comprises transforming a plant cell with a an isolated nucleic acid molecule encoding a cis-prenyltransferase under the control of suitable regulatory sequences; recovering a transformed plant cell produced; regenerating a plant from the transformed plant; and growing the transformed plant produced in step (c) under conditions wherein the isolated nucleic acid molecule encoding a cis-prenyltransferase is expressed and the growth phenotype of the transformed plant is altered. Genetically modified plants of this invention are fertile (i.e., capable of self- or cross-pollination with other plants of the same species to produce seed) and seeds so produced are capable of germination and growth. Furthermore, other plant properties are not affected in ways deleterious to agriculture, silviculture, horticulture, floriculture or cell culture, when the plants are transformed according to the present invention.

### DEFINITIONS

The following definitions are provided for the full understanding of terms and abbreviations used in this specification:

“Polymerase chain reaction” is abbreviated PCR.

“Open reading frame” is abbreviated ORF.

“Expressed sequence tag” is abbreviated EST.

“SDS polyacrylamide gel electrophoresis” is abbreviated SDS-PAGE.

“Polyisoprenoids” refer to a variety of hydrocarbons produced by plants that are built up of isoprene units (C₅H₈) (Tanaka, Y. In Rubber and Related Polyprenols. Methods in Plant Biochemistry; Dey, P. M. and Harborne, J. B., Eds., Academic Press: San Diego, Calif. (1991); Vol. 7, pp 519-536). Those with 45 to 115 carbon atoms and varying numbers of cis- and trans- (Z- and E-) double bonds are termed “polyprenols”, while those polyisoprenoids of longer chain length are termed natural “rubbers” (Tanaka, Y. In Minor Classes of Terpenoids. Methods in Plant Biochemistry; Dey, P. M. and Harborne, J. B., Eds., Academic: San Diego, Calif. (1991); Vol. 7, pp 537-542). There are several suggested functions for plant polyisoprenoids; however, the specific roles of the C₄₅-C₁₁₅ polyprenols remain unidentified (although as with most secondary metabolites they too most likely function in plant defense). Short-chain polyprenols may also be involved in protein glycosylation in plants, by analogy with the role of dolichols in animal metabolism.

The term “cis-prenyltransferase” refers generally to a class of enzymes (E.C. 2.5.1.31) capable of catalyzing the sequential addition of C₅ isopentenyl diphosphate (IPP) units to polyprenols and rubbers in cis 14 orientation. Two examples of cis-prenyltransferases are the undecaprenyl diphosphate synthase (EC 2.5.1.31) (Shimizu et al., J. Biol. Chem. 273:19476-19481 (1998); Apfel et al., J. Bacteriol. 181:483-492 (1999)) and yeast dehydrodolichyl diphosphate synthase (Sato et al., Mol. Cell. Biol. 19:471-483 (1999)).

The term “genetic modification” as used herein refers to the introduction of one or more exogeneous nucleic acid sequences, e.g., cis-prenyltransferase encoding sequences, as well as regulatory sequences, into one or more plant cells, which can generate whole, sexually competent, viable plants. The term “genetically modified plant” as used herein refers to a plant that has been generated through the aforementioned process. Genetically modified plants of the invention are capable of self-pollinating or cross-pollinating with other plants of the same species so that the foreign gene, carried in the germ line, can be inserted into or bred into useful plant varieties.

The term “altered growth phenotype” refers to a plant having a changed phenotype as relating to the growth of the plant. A plant will have an altered growth phenotype when it exhibits changes in growth of the total plant, specific tissues or organs of the plant, or the yield. Additionally, the term “altered growth phenotype” will encompass changes in the rate of development or size or characteristics of plant architecture.

The term “enhanced growth” is a concept well known to the person skilled in the art of plant biology and includes increased crop growth and/or enhanced biomass.

The term “maturity” in general refers to plants which have initiated the transition from vegetative to a reproductive phase of growth, but may also refer to fruit maturation or ripening.

The term “increased yield” or “increased plant yield” refers to an increase in harvestable material resulting from, for example, increased crop growth, increased biomass, or increased seed/fruit yield. Increases can result, for example, from an increased overall growth rate, increased root or tuber size, increased shoot growth, increased leaf biomass, and/or increased seed/fruit growth/number.

The term “plant architecture” refers to any trait of morphology of a plant. Structural features encompassed by the term may include shape, size, number, colour, texture, arrangement and patternation of any cell, tissue or organ or groups of cells, tissues, or organs of plants (e.g., shoots, roots, calli, tumors, flowers, leaves).

The term “plant” refers to a whole plant, a plant tissue, a plant organ, or a portion thereof. Plantlets are also included within the meaning of “plant”.

The term “plant tissue” or “plant organ” may refer to any part of a plant, including, but not limited to: the root, leaf, shoot, fruit, petiole, trichome, flower, sepals, petal, hypocotyl, stigma, style, stamen, pollen, ovule, seed, embryo, endosperm, seed coat, aleurone, fibre, nodule, cambium, wood, heartwood, parenchyma, sclerenchyma, seive element, phloem, or vascular tissue, or parts thereof.

The term “plant cell” as used herein refers to any cell of plant origin, including protoplasts, gamete-producing cells, and cells which regenerate into whole plants.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “fragment” refers to a DNA or amino acid sequence comprising a subsequence of a cis-prenyltransferase nucleic acid sequence or protein. However, an active fragment of the present invention comprises a sufficient portion of the protein to maintain activity.

A nucleic acid molecule is “hybridizable” to another nucleic acid molecule, such as a cDNA, genomic DNA, or RNA molecule, when a single-stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. (Molecular Cloning: A Laboratory Manual, 2^{nd} ed.; Cold Spring Harbor Laboratory: Cold Spring Harbor, N.Y., 1989 (hereinafter “Maniatis”), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washed with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Maniatus, supra, 9.50-9.51). For hybridizations with shorter nucleic acids, i.e., oligonucleotides, the position of mismatches becomes more important, and the length of the oligonucleotide determines its specificity (see Maniatus, supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “complementary” is used to describe the relationship between nucleotide bases that are capable of hybridizing to one another. For example, with respect to DNA, adenosine is complementary to thymine and cytosine is complementary to guanine.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including (but not limited to) those described in: 1.) Computational Molecular Biology; Lesk, A. M., Ed.; Oxford University: NY, 1988; 2.) Biocomputing: Informatics and Genome Projects: Smith, D. W., Ed.; Academic: NY, 1993; 3.) Computer Analysis of Sequence Data. Part I; Griffin, A. M., and Griffin, H. G., Eds.; Humana: NJ, 1994; 4.) Sequence Analysis in Molecular Biology; von Heinje, G., Ed.; Academic, 1987; and 5.) Sequence Analysis Primer; Gribskov, M. and Devereux, J., Eds.; Stockton: NY, 1991. Preferred methods to determine identity are designed to give the best match between the sequences tested. Methods to determine identity and similarity are codified in publicly available computer programs. Sequence alignments and percent identity calculations may be performed using the AlignX program of the Vector NTI bioinformatics computing suite (InforMax Inc., North Bethesda, Md.). Multiple alignment of the sequences was performed using the Clustal method of alignment (Higgins and Sharp, CABIOS. 5:151-153 (1989)) with the default parameters (GAP OPENING PENALTY=10, GAP EXTENSION PENALTY=0.1). Default parameters for pairwise alignments using the Clustal method were KTUPLE SIZE=1, GAP PENALTY=3, WINDOW SIZE=5 and NUMBER OF BEST DIAGONALS=5.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” or “exogenous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding sites, stem-loop structures, or any other gene expression control elements which are known to activate gene expression and/or increase the amount of gene products.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (Plant Cell, 1:671-680 (1989)).

“Promoter” refers to a nucleotide sequence, usually upstream (5′) to its coding sequence, capable of controlling the expression of a coding sequence by providing the recognition for RNA polymerase and other factors required for proper transcription. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further recognized that since in most cases the exact boundaries of regulatory sequences have not been completely defined, DNA fragments of different lengths may have identical promoter activity.

The term “promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes DNA sequences that are involved in the binding of protein factors which control the effectiveness of transcription initiation in response to physiological or developmental conditions. Additionally, “promoter” also refers to a nucleotide sequence that includes regulatory elements that are capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue-specificity of a promoter. Enhancers are capable of operating in both orientations (normal or flipped), and are capable of functioning even when moved either upstream or downstream from the promoter. Both enhancers and other upstream promoter elements bind sequence-specific DNA-binding proteins that mediate their effects.

“Constitutive promoter” refers to promoters that direct gene expression in all tissues and at all times. “Regulated promoter” refers to promoters that direct gene expression not constitutively but in a temporally- and/or spatially-regulated manner and include tissue-specific, developmental stage-specific, and inducible promoters. It includes natural and synthetic sequences, as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered; numerous examples may be found in the compilation by Okamuro et al. (Biochemistry of Plants 15:1-82 (1989); see also WO 00/56905, Tables 3-4). Since in most cases the exact boundaries of regulatory sequences have not been completely defined, DNA fragments of different lengths may have identical promoter activity. Typical regulated promoters useful in plants include, but are not limited to: safener-inducible promoters, promoters derived from the tetracycline-inducible system, promoters derived from salicylate-inducible systems, promoters derived from alcohol-inducible systems, promoters derived from glucocorticoid-inducible systems, promoters derived from pathogen-inducible systems, and promoters derived from ecdysome-inducible systems.

“Tissue-specific promoter” refers to regulated promoters that are not expressed in all plant cells but only in one or more cell types in specific organs (e.g., leaves, shoot apical meristem, flower, or seeds), specific tissues (e.g., embryo or cotyledon), or specific cell types (e.g., leaf parenchyma, pollen, egg cell, microspore- or megaspore mother cells, or seed storage cells). These also include “developmental-stage specific promoters” that are temporally regulated, such as in early or late embryogenesis, during fruit ripening in developing seeds or fruit, in fully differentiated leaf, or at the onset of senescence. It is understood that the developmental specificity of the activation of a promoter (and, hence, of the expression of the coding sequence under its control) in a transgene may be altered with respect to its endogenous expression. For example, when a transgene under the control of a floral promoter is transformed into a plant, even when it is the same species from which the promoter was isolated, the expression specificity of the transgene will vary in different transgenic lines due to its insertion in different locations of the chromosomes.

“Plant developmental stage-specific promoter” refers to a promoter that is expressed not constitutively but at a specific plant developmental stage or stages. Plant development goes through different stages; for example, in the context of this invention, the germine goes through different developmental stages starting, say, from fertilization through development of embryo, vegetative shoot apical meristem, floral shoot apical meristem, anther and pistil primordia, anther and pistil, micro- and macrospore mother cells, and macrospore (egg) and microspore (pollen).

“Inducible promoter” refers to those regulated promoters that can be turned on in one or more cell types by a stimulus external to the plant, such as a chemical, light, hormone, stress, or a pathogen.

“Promoter activation” means that the promoter has become activated (or turned “on”) so that it functions to drive the expression of a downstream genetic element. Constitutive promoters are continually activated. A regulated promoter may be activated by virtue of its responsiveness to various external stimuli (inducible promoter), or developmental signals during plant growth and differentiation, such as tissue specificity (floral-specific, anther-specific, pollen-specific, seed-specific, etc.) and development-stage specificity (vegetative-specific or floral-, shoot-, or apical meristem-specific, male germline-specific, female germline-specific, etc).

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences can be operably-linked to regulatory sequences in sense or antisense orientation. “Unlinked” means that the associated genetic elements are not closely associated with one another and the function of one does not affect the other.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense RNA” refers to RNA transcript that includes the mRNA and so can be translated into protein by the cell. “Antisense RNA” refers to RNA transcript that is complementary to all or part of a target primary transcript or mRNA and that blocks the expression of a target gene (U.S. Pat. No. 5,107,065; WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “expression”, as used herein, refers to the transcription and stable accumulation of sense (mRNA) or antisense RNA derived from the nucleic acid fragments of the present invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020).

“Constitutive expression” refers to expression using a constitutive or regulated promoter. “Conditional” and “regulated expression” refer to expression controlled by a regulated promoter. “Transient” expression in the context of this invention refers to expression only in specific developmental stages or tissue in one or two generations. “Non-specific expression” refers to constitutive expression or low level, basal (‘leaky’) expression in nondesired cells, tissues, or generations.

The term “altered biological activity” will refer to an activity, associated with a protein encoded by a nucleotide sequence which can be measured by an assay method, where that activity is either greater than or less than the activity associated with the native sequence. “Enhanced biological activity” refers to an altered activity that is greater than that associated with the native sequence. “Diminished biological activity” is an altered activity that is less than that associated with the native sequence.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be (but are not limited to) intracellular localization signals.

The term “sporophyte” means the diploid phase or cells of a plant.

The term “gametophyte” means the haploid phase or cells of a plant. This is the stage in a plant's life cycle between meiosis and fertilization. The male gametophyte includes the haploid phase or cells of the pollen and the female gametophyte includes the haploid phase or cells of the egg cell.

The term “plant life cycle” means a complete sequence of developmental events in the life of a plant, such as from fertilization to the next fertilization or from flowering in one generation to the next. The term “generation” means a plant life cycle starting from fertilization to fertilization.

“Primary transformant” and “T₀ generation” refer to transgenic plants that are of the same genetic generation as the tissue which was initially transformed (i.e., not having gone through meiosis and fertilization since transformation).

“Secondary transformants” and the “T₁, T₂, T₃, etc. generations” refer to transgenic plants derived from primary transformants through one or more meiotic and fertilization cycles. They may be derived by self-fertilization of primary or secondary transformants or by crosses of primary or secondary transformants with other transformed or untransformed plants.

“Transformation” refers to the transfer of a nucleic acid fragment into the genome of a host organism, resulting in genetically stable inheritance. The polynucleotide may be transiently or stably introduced into the host cell and may be maintained in a non-integrated fashion (e.g., as a plasmid) or alternatively, may be integrated into the host genome. The resulting transformed plant cell or plant tissue can then be used to regenerate a transformed plant in a manner known by a skilled person. Host organisms containing the transformed nucleic acid fragments are referred to as “transgenic” or “recombinant” or “transformed” organisms.

“Regeneration” means growing a whole plant from a plant cell, a group of plant cells, a plant part (including seeds), or a plant piece (e.g., from a protoplast, callus, or tissue part).

The terms “plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements (in addition to the foreign gene) that facilitate transformation of a particular host cell. “Expression cassette” refers to a specific vector containing a foreign gene and having elements in addition to the foreign gene that allow for enhanced expression of that gene in a foreign host.

“Marker” refers to a gene encoding a trait or a phenotype which permits the selection of, or the screening for, a plant or plant cell containing the marker.

The term “conserved domain” means a set of amino acids conserved at specific positions along an aligned sequence of evolutionarily related proteins. While amino acids at other positions can vary between homologous proteins, amino acids that are highly conserved at specific positions indicate amino acids that are essential in the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family. Conserved domains are specifically described for the family of cis-prenyltransferases, according to the work of Apfel, C. M. et al. (J. Bact. 181(2): 483-492 (1999)) and Hallahan and Keiper-Hrynko (PCT/US03/36164).

The term “non-conserved domain” means a set of amino acids, present between conserved domains, which whilst the individual amino acids are not conserved at specific positions along an aligned sequence of evolutionarily related proteins, is recognizable by its presence or absence in aligned sequences of evolutionary related proteins. The presence of such a domain, despite positional non-conservation among its constituent amino acids, indicates that the domain plays a role essential in the structure, the stability, or the activity of a protein, e.g., by increasing the distance between other (conserved) domains. Because they are identified by their presence in aligned sequences of a family of protein homologues, they can be used as identifiers, or “signatures”, to determine if a protein with a newly determined sequence belongs to a previously identified protein family or subfamily.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol. 215:403-410 (1990); 3.) Vector NTI (InforMax Inc., North Bethesda, Md.); and 4.) DNASTAR (DNASTAR Inc., Madison, Wis.). Within the context of this application it will be understood that where sequence analysis software is used for analysis, that the results of the analysis will be based on the “default values” of the program referenced, unless otherwise specified. As used herein “default vales” will mean any set of values or parameters which originally load with the software when first initialized.

Standard recombinant DNA and molecular cloning techniques used herein are well known in the art and are described by: Sambrook, J., Fritsch, E. F. and Maniatis, T., Molecular Cloning: A Laboratory Manual; 2nd ed.; Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions; Cold Spring Harbor Laboratory: Cold Spring Harbor, N.Y. (1984); and by Ausubel, F. M. et al., Current Protocols in Molecular Biology; Greene Publishing Assoc. and Wiley-Interscience (1987).

### Polyisoprenoids

Plants synthesize a variety of hydrocarbons built up of isoprene units (C₅H₈), termed polyisoprenoids (Tanaka, Y. In Rubber and Related Polyprenols. Methods in Plant Biochemistry, Dey, P. M. and Harborne, J. B., Eds., Academic: San Diego, Calif. (1991); Vol. 7, pp 519-536). Representative polyisoprenoid structures are shown below for:

1. Dolichols (n=12-18):

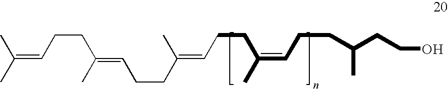

2. 3-trans, poly-cis polyprenols (e.g., ficaprenols, n=10-12):

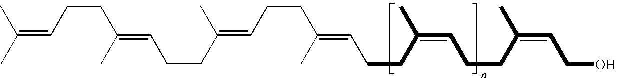

3. 2-trans, poly-cis polyprenols (e.g., betulaprenols, n=6-9)

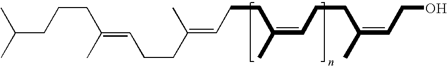

The left-handed portion of each of the molecules above are formed from allylic terpenoid diphosphate initiators (e.g., dimethylallyldiphosphate (DMAPP; C₅), geranyl diphosphate (GPP; C₁₀), farnesyl diphosphate (FPP; C₁₅), and geranylgeranyl diphosphate (GGPP; C₂₀)). The remaining portion of the molecules shown with heavy-set lines are formed by the activity of cis-prenyltransferases catalyzing sequential additions of isopentenyl diphosphate (IPP; C₅)). In general, those polyisoprenoids with 45 to 115 carbon atoms, and varying numbers of cis- and trans- (Z- and E-) double bonds, are termed polyprenols, while those of longer chain length are termed rubbers (Tanaka, Y. In Minor Classes of Terpenoids. Methods in Plant Biochemistry; Dey, P. M. and Harborne, J. B., Eds., Academic: San Diego, Calif. (1991); Vol. 7, pp 537-542).

There are several suggested functions for plant polyisoprenoids. Terpenoid quinones are most likely involved in photophosphorylation and respiratory chain phosphorylation. Rubbers have been implicated in plant defense against herbivory, possibly serving to repel and entrap insects and seal wounds in a manner analogous to plant resins. The specific roles of the C₄₅-C₁₁₅ polyprenols remain unidentified; although, as with most secondary metabolites, they too are thought most likely to function in plant defense. Short-chain polyprenols may also be involved in protein glycosylation in plants, by analogy with the role of dolichols in animal metabolism. In no case has a role for these secondary metabolites in modulating plant development been proposed.

### Cis-Prenyltransferases

Cis-prenyltransferases are a family of enzymes that are responsible for synthesizing plant polyisoprenoids (specifically polyprenols and natural rubbers), by catalyzing the sequential addition of IPP to an initiator molecule in head-to-tail condensation reactions. The initiator molecules themselves are derived from isoprene units through the action of distinct prenyltransferases.

Cis-prenyltransferases are ˜30 kD proteins. The expression of full-length plant cis-prenyltransferase cDNAs yields a mature protein capable of the synthesis of cis-polyisoprenoids from IPP as the substrate. Cis-prenyltransferases were previously known to play a vital role in cellular activity, the biosynthesis of plant cell walls, and posttranslational glycosylation of proteins. In the present invention, the roles of cis-prenyltransferases have been expanded to further include their ability to affect plant growth and development.

The identification of genes encoding the bacterial cis-prenyltransferase undecaprenyl diphosphate synthase (di-trans,poly-cis-decaprenylcistransferase, or Upp synthetase; EC 2.5.1.31) (Shimizu et al., J. Biol. Chem. 273:19476-19481 (1998); Apfel et al., J. Bacteriol. 181:483-492 (1999)) and yeast dehydrodolichyl diphosphate (Dedol-PP) synthase (Sato et al., Mol. Cell. Biol. 19:471-483 (1999)) have facilitated the identification of prenyltransferases that condense isoprene units in a cis-configuration in other organisms. This was, in part, enabled by the publication of Apfel et al. (supra) of an alignment of the deduced amino acid sequence of the E. coli Upp synthase gene with a number (28) of other publicly-available sequences from bacteria, yeast (Saccharomyces cerevisiae) and one eukaryote (Caenorhabditis elegans), which revealed five conserved domains. Four of these domains are included herein as SEQ ID Nos 7-10.

The authors predicted that these conserved domains, as well as a few single conserved amino acids outside of the conserved domains, likely represented the active site of the protein.

Since the work of Apfel et al. (supra), U.S. Pat. No. 6,645,747 taught the identification and characterization of cis-prenyltransferase proteins from wheat, grape, soybean, rice, African daisy, rubber tree (Hevea brasiliensis) and pot marigold (see also GenBank Accession Numbers: AY124934, AY124474, AY124473, AY124472, AY124471, AY124470, AY124469, AY124468, AY124467, AY124466, AY124465, AY124464). Additional cis-prenyltransferases have been isolated from H. brasiliensis by Asawatreratanakul, K. et al. (AB061236) and Sando, T. et al. (AB074307) and from Arabidopsis by Oh, S. K. et al. (AF162441).

Furthermore, available knowledge concerning cis-prenyltransferases was further advanced by Hallahan and Keiper-Hrynko (PCT/US03/36164), with: 1.) the isolation of cis-prenyltransferase cDNAs from the natural rubber-producing plants russian dandelion (Taraxacum kok-saghyz) and sunflower (Helianthus annus); and 2.) the description of modified sequences of conserved Domains I (SEQ ID NO:11), Domain IV (SEQ ID NO:12), and Domain V (SEQ ID NO:13), with respect to Apfel et al. (supra), that are indicative of the subfamily of cis-prenyltransferases associated with rubber-producing plants and the presence of a unique non-conserved domain between conserved domain IV and V, that is present in cis-prenyltransferases from rubber-producing plants and that is absent in cis-prenyltransferases from other plants.

In one embodiment of the invention, most preferred cis-prenyltransferase proteins are those from rubber (Hevea brasiliensis), rice, and soybean, (SEQ ID NOs:2, 4, and 6 respectively) and a newly identified cis-prenyltransferase homolog from Arabidopsis, Apt5 (SEQ ID NO:21). However, it will be obvious to one of skill in the art that a variety of cis-prenyltransferase genes and their homologs would likely be suitable for the purposes of the invention herein. The source from which these cis-prenyltransferase genes are derived (e.g., microbial, plant, animal etc.) is not limiting to the invention herein. Thus, those nucleic acids containing significant homology to Domain I (SEQ ID NO:7), Domain II (SEQ ID NO:8), Domain III (SEQ ID NO:9), and Domain V (SEQ ID NO:10), as described by Apfel et al. (supra) or the modified domains described by Hallahan and Keiper-Hrynko (PCT/US03/36164) (SEQ ID NOs: 11-13), would be expected to convey a similar phenotype of modified growth to maturity and/or yield and/or architecture for plants transformed with these sequences.

In an alternative embodiment of the invention, suitable nucleic acids useful in the methods described herein encode polypeptides having cis-prenyltransferase activity, wherein the polypeptide is capable of catalyzing the sequential addition of IPP units to polyprenols and rubbers in cis 1-4 orientation. In a preferred embodiment, suitable nucleic acids useful for the purposes described herein are at least about 70% identical, preferably at least about 80% identical to the Hpt2, Spt1, Rpt1, and/or Apt5 amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

### Recombinant Expression in Plants

It was unexpected that introduction of chimeric genes encoding the instant cis-prenyltransferase enzymes, under the control of the appropriate promoters, would produce a phenotype of modified growth to maturity. While not intending to suggest a mechanism for the present invention, it may be that, given the involvement of IPP as a precursor of many classes of plant hormones (i.e., gibberellins, brassinosteroids, cytokinins and abscissic acid), perturbations in IPP flux may result in altered hormone biosynthesis in the transgenic plants. And yet, although the phenomenum is not clearly understood, it is contemplated that it will be useful to overexpress cis-prenyltransferase genes both in natural host cells as well as heterologous plant hosts.

In some applications, it might be desirable to express the cis-prenyltransferases in specific plant tissues and/or cell types (e.g., to modify fruit or seed production, manipulate the strength/thickness of a stem, etc.), or during developmental stages in which they would normally not be encountered.

Alternatively, the constitutive over-expression of a cis-prenyltransferase in a transformed plant could modify the plant's overall growth rate to maturity. When the result of such over-expression was to produce a phenotype characterized with an accelerated growth rate to maturity, this would be desirable when modifying the plant such that it would be capable of maturing in shorter growing seasons, thus permitting expansion of the geographic range in which these plants grew. Additionally, an overall increase in growth rate to maturity could provide significant economic advantages to the grower (e.g., in silviculture, cell culture, etc.).

Genetically modified plants of the present invention are produced by overexpression of the instant cis-prenyltransferases. Generally, this may be accomplished by first constructing chimeric genes in which the cis-prenyltransferase coding region is operably-linked to control sequences capable of directing expression of the gene in the desired tissues at the desired stage of development. These control sequences may comprise a promoter, enhancer, silencer, intron sequences, 3′UTR and/or 5′UTR regions, protein and/or RNA stabilizing elements. For reasons of convenience, the chimeric genes may comprise promoter sequences and translation leader sequences derived from the same genes. 3′ Non-coding sequences encoding transcription termination signals must also be provided. It is preferred that the chimeric gene be introduced via a vector and that the vector harboring the cis-prenyltransferase sequence also contain one or more selectable marker genes so that cells transformed with the chimeric gene can be selected from non-transformed cells.

### Promoters

The present invention makes use of a variety of plant promoters to drive the expression of the chimeric genetic sequence comprising a cis-prenyltransferase gene or functional fragment thereof.

Regulated expression of cis-prenyltransferase expression is possible by placing the cis-prenyltransferase under the control of promoters that may be conditionally regulated. Any promoter functional in a plant will be suitable, including (but not limited to): constitutive plant promoters, plant tissue-specific promoters, plant development-stage specific promoters, inducible plant promoters, viral promoters, male germline-specific promoters, female germline-specific promoters, flower-specific promoters, and vegetative shoot apical meristem-specific promoters.

Some suitable examples of constitutive promoters include those from nopaline synthase (nos), octopine synthase (ocs), cauliflower mosaic virus (CaMV) (35S [Odell et al., Nature, 313: 810-812 (1985)] and 19S [Nilsson et al., Physiol. Plant. 100:456-462 (1997)]), actin (McElroy et al., Plant Cell, 2:163-171 (1990)), actin 2 (An et al., Plant J. 10(1):107-121 (1996)) and ubiquitin (Christensen et al., Plant Mol. Biol. 18: 675-689 (1992)) genes.

Several tissue-specific regulated genes and/or promoters have been reported in plants. These include genes encoding: 1.) the seed storage proteins (e.g., napin, cruciferin, □-conglycinin, and phaseolin); zein or oil body proteins (e.g., oleosin); genes involved in fatty acid biosynthesis (e.g., acyl carrier protein, stearoyl-ACP desaturase, and fatty acid desaturases (fad 2-1)); and 4.) other genes expressed during embryo development (e.g., Bce4 [see, for example, EP 255378 and Kridl et al., Seed Science Research 1:209-219 (1991)]). Particularly useful for seed-specific expression is the pea vicilin promoter (Czako et al., Mol. Gen. Genet. 235(1): 3340 (1992)). Other useful promoters for expression in mature leaves are those that are switched on at the onset of senescence, such as the SAG promoter from Arabidopsis (Gan et al., Science (Washington, D.C.) 270(5244): 1986-8 (1995)).

A class of fruit-specific promoters expressed at or during anthesis through fruit development, at least until the beginning of ripening, is discussed in U.S. Pat. No. 4,943,674, the disclosure of which is hereby incorporated by reference. cDNA clones that are preferentially expressed in cotton fiber have been isolated (John et al., Proc. Natl. Acad. Sci. U.S.A. 89(13): 5769-73 (1992)). cDNA clones from tomato displaying differential expression during fruit development have been isolated and characterized (Mansson et al., Mol. Gen. Genet. 200:356-361 (1985); Slater et al., Plant Mol. Biol. 5:137-147 (1985)). The promoter for polygalacturonase gene is active in fruit ripening. The polygalacturonase gene is described in the following U.S. patents, which disclosures are incorporated herein by reference: U.S. Pat. No. 4,535,060, U.S. Pat. No. 4,769,061, U.S. Pat. No. 4,801,590, and U.S. Pat. No. 5,107,065.

Mature plastid mRNA for psbA (one of the components of photosystem II) reaches its highest level late in fruit development, in contrast to plastid mRNAs for other components of photosystem I and II which decline to nondetectable levels in chromoplasts after the onset of ripening (Piechulla et al., Plant Mol. Biol. 7:367-376 (1986)). Recently, cDNA clones representing genes apparently involved in tomato pollen (McCormick et al., Tomato Biotechnology, Alan R. Liss: New York (1987)) and pistil (Gasser et al., Plant Cell 1:15-24 (1989)) interactions have also been isolated and characterized.

Other examples of tissue-specific promoters include those that direct expression in leaf cells following damage to the leaf (e.g., from chewing insects), in tubers (e.g., patatin gene promoter), and in fiber cells. One example of a developmentally-regulated fiber cell protein is E6 (John et al., Proc. Natl. Acad. Sci. U.S.A. (89(13): 5769-73 1992)); the E6 gene is most active in fiber, although low levels of transcripts are found in leaf, ovule and flower.

Although the promoters described above are provided for the purposes of exemplification only, the present invention is not to be limited by those provided therein. Those skilled in the art will readily be in a position to provide additional tissue-specific promoters that are useful in performing the present invention (see, for example U.S. Pat. No. 5,589,379) which are:
1. stem-specific (e.g., to modify strength and thickness of a plant stem [wherein increased strength and thickness can confer improved stability and wind-resistance]);
2. meristem-specific (e.g., to modify apical dominance or the “bushiness” of a plant);
3. tuber-specific (e.g., to modify tuber production);
4. seed-specific (e.g., to modify seed production in plants [wherein increased seed production can be quantitated as increased seed set and/or seed production and/or seed yield);
5. endosperm-specific (e.g., to modify grain yield, since grain yield in crop plants is largely a function of the amount of starch produced in the endosperm of the seed);
6. root-specific (e.g., to modify the production of roots or storage organs derived from roots);
7. nodule-specific (e.g., to modify the nitrogen-fixing capability of a plant);
8. embryo-specific (e.g., to modify embryo size, which is important for growth after germination); and
9. leaf-specific, flower-specific, or fruit-specific.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., Plant Cell, 9:1527-1545 (1997)).

Germine specific promoters, responsive to male, female, or both male-female specific cell lineages are also useful in the present invention. For instance, transgenes can be expressed or removed from pollen by site-specific recombinase expression under the control of male germline-specific genes in anther primordia genes (e.g., Arabidopsis Apetalla 3 and Pistilata (PI) or their orthologs from other plant species), in sporophytic anther tissue (e.g., Bcp I and TA29 promoters) or gametophytic pollen. Similarly, transgenes can be expressed or removed from ovules by site-specific recombinase expression under the control of female germline-specific genes in ovule primordia. Transgenes can be expressed or removed from both male- and female-specific germlines by expression of an active site-specific recombinase gene under the control of a promoter for genes common to both male and female lineages in flower (e.g., Arabidopsis agamous gene or its orthologs in other species), in floral meristem (e.g., Arabidopsis Apetala 1, Leafy, and Erecta or their orthologs from other species), and in vegetative shoot apical meristem (such as Arabidopsis WUSCHEL (WUS) and SHOOT MERISTEMLESS (STM) or their orthologs from other species). Promoters of shoot apical meristem are especially useful for removing or expressing transformation marker genes early in tissue-culture following selection or in planta following a transformation phenotype.

Similarly, several inducible promoters (“gene switches”) have been reported. Many are described in the reviews by Gatz (Current Opinion in Biotechnology, 7:168-172 (1996); Annu. Rev. Plant Physiol. Plant Mol. Biol. 48: 89-108 (1997)). These include tetracycline repressor systems, Lac repressor systems, copper-inducible systems, salicylate-inducible systems (e.g., the PR1a system), and glucocorticoid- (Aoyama T. et al., N-H Plant Journal 11:605-612 (1997)) and ecdysome-inducible systems. Also included are the benzene sulphonamide- (U.S. Pat. No. 5,364,780) and alcohol- (WO 97/06269 and WO 97/06268) -inducible systems and glutathione S-transferase promoters. Other studies have focused on genes inducibly regulated in response to environmental stress or stimuli such as increased salinity, drought, pathogen, and wounding (Graham et al., J. Biol. Chem. 260:6555-6560 (1985); Graham et al., J. Biol. Chem. 260:6561-6554 (1985); Smith et al., Planta 168:94-100 (1986)). Accumulation of a metallocarboxypeptidase-inhibitor protein has been reported in leaves of wounded potato plants (Graham et al., Biochem Biophys Res Comm 101:1164-1170 (1981)). Other plant genes that have been reported to be induced include: methyl jasmonate, elicitors, heat-shock, anerobic stress, or herbicide safeners Expression Vectors

Plasmid vectors comprising the chimeric cis-prenyltransferase genes can then be constructed. The choice of a plasmid vector depends upon the method that will be used to transform host plants. The skilled artisan is well aware of the genetic elements that must be present on the plasmid vector in order to successfully transform, select and propagate host cells containing the chimeric gene.

Methods which are well known to those skilled in the art can be used to construct various plasmids and vectors; see, for example, the techniques described in Sambrook et al., Molecular Cloning: A Laboratory Manual, 2^{nd} ed., Cold Spring Harbor Laboratory: Cold Spring Harbor, N.Y., 1989 (hereinafter “Maniatus”); and by Ausubel et al., Current Protocols in Molecular Biology, published by Greene Publishing Assoc. and Wiley-Interscience, 1987.

A preferred vector of the invention is an expression vector that provides for expression of a cis-prenyltransferase coding sequence in the selected host. Expression vectors can for instance be cloning vectors, binary vectors or integrating vectors. Expression comprises transcription of the nucleic acid molecule preferably into a translatable mRNA. Regulatory elements ensuring expression in eukaryotic cells are well known to those skilled in the art. In the case of eukaryotic cells, they normally comprise promoters ensuring initiation of transcription and optionally poly-A signals ensuring termination of transcription and stabilization of the transcript (for example, those of the 35S RNA from Cauliflower Mosaic Virus (CaMV)). The termination signals usually employed are from the Nopaline Synthase promoter or from the CAMV 35S promoter. A plant translational enhancer often used is the tobacco mosaic virus (TMV) omega sequences; additionally, the inclusion of an intron (e.g., Intron-1 from the Shrunken gene of maize) has been shown to increase expression levels by up to 100-fold (Mait, Transgenic Res. 6:143-156 (1997); Ni, Plant Journal 7:661-676 (1995)). Additional regulatory elements may include transcriptional as well as translational enhancers.

In addition to the elements described above for a preferred expression vector, it is also useful for the vector to comprise a selectable and/or scorable marker. Preferably, the marker gene is an antibiotic resistance gene whereby the appropriate antibiotic can be used to select for transformed cells from among cells that are not transformed. Selectable marker genes useful for the selection of transformed plant cells, callus, plant tissue and plants are well known to those skilled in the art. Examples include, but are not limited to: npt, which confers resistance to the aminoglycosides neomycin, kanamycin and paromycin; hygro, which confers resistance to hygromycin; trpB, which allows cells to utilize indole in place of tryptophan; hisD, which allows cells to utilize histinol in place of histidine (Hartman, Proc. Natl. Acad. Sci. USA 85:8047 (1988)); mannose-6-phosphate isomerase, which allows cells to utilize mannose (WO 94/20627); ODC (ornithine decarboxylase), which confers resistance to the ornithine decarboxylase inhibitor, 2-(difluoromethyl)-DL-ornithine, DFMO (McConlogue, In: Current Communications in Molecular Biology, Cold Spring Harbor Laboratory: Cold Spring Harbor, N.Y. (1987)); and deaminase from Aspergillus terreus, which confers resistance to Blasticidin S (Tamura, Biosci. Biotechnol. Biochem. 59 2336-2338 (1995)).

Useful scorable markers are also known to those skilled in the art and are commercially available, such as the genes encoding luciferase (Giacomin, Pl. Sci. 116:59-72 (1996); Scikantha, J. Bact. 178:121 (1996)), green fluorescent protein (Gerdes, FEBS Lett. 389:44-47 (1996)) or R-glucuronidase (Jefferson, EMBO J. 6:3901-3907 (1987)). This embodiment is particularly useful for simple and rapid screening of cells, tissues and organisms containing a vector comprising a cis-prenyltransferase.

For some applications it may be useful to direct the cis-prenyltransferase proteins to different cellular compartments. It is thus envisioned that the chimeric genes described above may be further modified by the addition of appropriate intracellular targeting sequences to their coding regions (and/or with targeting sequences that are already present removed). These additional targeting sequences include chloroplast transit peptides (Keegstra et al., Cell 56:247-253 (1989)), signal sequences that direct proteins to the endoplasmic reticulum (Chrispeels et al., Ann. Rev. Plant Phys. Plant Mol. 42:21-53 (1991)), and nuclear localization signals (Raikhel et al., Plant Phys. 100:1627-1632 (1992)). While the references cited give examples of each of these, the list is not exhaustive and more targeting signals of utility may be discovered in the future which are useful in the invention.

### Transformation Methods

A variety of techniques are available and known to those skilled in the art for introduction of constructs into a plant cell host. These techniques include transformation with DNA employing Agrobacterium tumefaciens or A. rhizogenes as the transforming agent. It is particularly preferred to use the binary type vectors of Ti and Ri plasmids of Agrobacterium spp. Ti-derived vectors transform a wide variety of higher plants, including monocotyledonous and dicotyledonous plants such as soybean, cotton, rape, tobacco, and rice (Pacciotti et al., Bio/Technology 3:241 (1985); Byrne et al., Plant Cell, Tissue and Organ Culture 8:3 (1987); Sukhapinda et al., Plant Mol. Biol. 8:209-216 (1987); Lorz et al., Mol. Gen. Genet. 199:178 (1985); Potrykus, Mol. Gen. Genet. 199:183 (1985); Park et al., J. Plant Biol. 38(4):365-71 (1995); Hiei et al., Plant J. 6:271-282 (1994)). The use of T-DNA to transform plant cells has received extensive study and is amply described (EP 120516; Hoekema, In: The Binary Plant Vector System, Offset-drukkerij Kanters B.V.; Alblasserdam (1985), Chapter V; Knauf et al., Genetic Analysis of Host Range Expression by Agrobacterium, In: Molecular Genetics of the Bacteria-Plant Interaction, Puhler, A. Ed.; Springer-Verlag: New York, 1983, p 245; and An et al., EMBO J. 4:277-284 (1985)). For introduction into plants, the chimeric genes of the invention can be inserted into binary vectors as described in the examples.

Other transformation methods are available to those skilled in the art, such as: 1.) direct uptake of foreign DNA constructs (see EP 295959); 2.) techniques of electroporation (see Fromm et al., Nature (London) 319:791 (1986)); 3.) high-velocity ballistic bombardment with metal particles coated with the nucleic acid constructs (see Kline et al., Nature (London) 327:70 (1987), and see U.S. Pat. No. 4,945,050); or 4.) microinjection (see Gene Transfer To Plants, Potrykus and Spangenberg, Eds., Springer Verlag: Berlin, N.Y. (1995)). The transformation of most dicotyledonous plants is possible with the methods described above; however, additional transformation techniques have been developed for the successful transformation of monocotyledonous plants. These include protoplast transformation and transformation by an in planta method using Agrobacterium tumefaciens. This in planta method (Bechtold and Pelletier, C. R. Acad. Sci. Paris, 316:1194 (1993); or Clough S. J., Bent A. F.; Plant Journal 16(6): 735-43 (1998)) involves the application of A. tumefaciens to the outside of the developing flower bud and then introduction of the binary vector DNA to the developing microspore and/or macrospore and/or developing seed, so as to produce a transformed seed without the exogenous application of cytokinin and/or gibberellin. Those skilled in the art will be aware that the selection of tissue for use in such a procedure may vary; however, it is preferable generally to use plant material at the zygote formation stage for in planta transformation procedures.

Once transformed, the plant cells can be regenerated by those skilled in the art. Of particular relevance are the recently described methods to transform foreign genes into commercially important crops, such as rapeseed (see De Block et al., Plant Physiol. 91:694-701 (1989)), sunflower (Everett et al., Bio/Technology 5:1201 (1987)), soybean (McCabe et al., Bio/Technology 6:923 (1988); Hinchee et al., Bio/Technology 6:915 (1988); Chee et al., Plant Physiol. 91:1212-1218 (1989); Christou et al., Proc. Natl. Acad. Sci. USA 86:7500-7504 (1989); EP 301749), rice (Hiei et al., supra), corn (Gordon-Kamm et al., Plant Cell 2:603-618 (1990); Fromm et al., Biotechnology 8:833-839 (1990)), and Hevea (Yeang, H. Y., et al., Rubber Latex as an Expression System for High-value Proteins. In, Engineering Crop Plants for Industrial End Uses. Shewry, P. R., Napier, J. A., David, P. J., Eds.; Portland: London, 1998; pp 55-64).

Transgenic plant cells are then placed in an appropriate selective medium for selection of transgenic cells that are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic such as kanamycin, G418, bleomycin, hygromycin, chloramphenicol, herbicide, or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region that is not native to the gene from which the transcription-initiation-region is derived.

One skilled in the art recognizes that the expression level and regulation of a transgene in a plant can vary significantly from line to line. Thus, one has to test several lines to find one with the desired expression level and regulation. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA blots (Southern, J. Mol. Biol. 98: 503 (1975)), Northern analysis of mRNA expression (Kroczek, J. Chromatogr. Biomed. Appl., 618(1-2): 133-145 (1993)), Western analysis of protein expression, or phenotypic analysis. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

### Preferred Plant Hosts

In general, plants that can be manipulated according to the invention to display a modified growth phenotype (in terms of rate of growth to maturity, and/or yield and/or architecture) can be derived from any desired plant species that will support expression of a cis-prenyltransferase. Plants so transformed can be monocotyledonous plants or dicotyledonous plants, and preferably they belong to plant species of interest in agriculture, silviculture or horticulture (e.g., a crop plant, root plant, oil-producing plant, wood producing plant, agricultural plant, fodder or forage legume, companion plant, or horticultural plant).

Suitable plant species include, but are not limited to: those plant species which produce natural rubber (e.g., Hevea brasiliensis, Taraxacum spp., Parthenium argentatum), tobacco (Nicotiana spp.), tomato (Lycopersicon spp.), potato (Solanum spp.), hemp (Cannabis spp.), sunflower (Helianthus spp.), sorghum (Sorghum vulgare), wheat (Triticum spp.), maize (Zea mays), rice (Oryza sativa), rye (Secale cereale), oats (Avena spp.), barley (Hordeum vulgare), rapeseed (Brassica spp.), broad bean (Vicia faba), french bean (Phaseolus vulgaris), other bean species (Vigna spp.), lentil (Lens culinaris), soybean (Glycine max), arabidopsis (Arabidopsis thaliana), cotton (Gossypium hirsutum), petunia (Petunia hybrida), flax (Linum usitatissimum), carrot (Daucus carota sativa), tea, celery, brussel sprout, artichoke, okra, squash, kale, asparagus (Asparagus), banana (Musa), blueberry (Vaccinium), cacao (Theobroma), capsicum pepper (Capsicum), cassaya (Manihof), cucumber (Cucumis), eggplant (Solanum), lettuce (Lactuca), mango (Mangifera), oilseed rape, canola, cabbage, broccoli, cauliflower (Brassica), onions (Allium), papaya (Carica), peas (Pisum), peanut (Arachis), pineapple (Ananas), pinto bean, mung bean, pumpkin, zucchini (Cucurbita), radish (Raphanus), sesame (Sesame), spinach (Spinaceae), sorphum (Sorphum), strawberry (Fragana), sugarcane (Saccharum), sugar beet (Beta), sweet potato (Ipomoea), watermelon (Citrullus), yam (Dioscorea), alfalfa (Medicago), amaranth (Amaranthus), angelica (Agelica), castorbean (Ricinus), colewort (Crambe), jojoba (Simmondsia), jute (Corchorus), kenaf (Hibiscus), lupine (Lupinus), plantain (Plantago), sisal (Agave), snapdragon (Antirrhinum), switch grass (Panicum), apple (Malus), acacia (Acacia), chestnut (Castanea), citrus (Citrus), coconut (Cocos), coffee (Coffea), cypress (Cupressus), eucalypti (Eucalyptus), grape (Vitis), hemlock (Tsuga), hickory (Carya), maple (Acer), oak (Quercus), pear (Pyrus), peach, plum, cherry (Prunus), pine (Pinus), poplar (Populus), rose (Rosa), spruce (Picea), and walnut (Juglans).

Phenotype of Plants Expressing cis-Prenyltransferases

The present invention provides a method for manipulating the rate of growth to maturity and/or yield and/or architecture of a genetically modified plant, as compared to a plant of the same species not genetically modified (i.e., a ‘wild-type’ plant). This method relies on elevating the expression of cis-prenyltransferase gene(s) in the plant. Thus, the method comprises:
a) transforming a plant cell with a an isolated nucleic acid molecule encoding a cis-prenyltransferase under the control of suitable regulatory sequences;
b) recovering a transformed plant cell produced in step (a);
c) regenerating a plant from the transformed plant cell of step (b); and
d) growing the transformed plant produced in step (c) under conditions wherein the isolated nucleic acid molecule encoding a cis-prenyltransferase is expressed and the growth phenotype of the transformed plant is altered.

“Plant architectural trait” refers to the general morphology or trait of a plant including (but not limited) to any one of the structural features provided as examples below: shape, size, number, colour, texture, arrangement and patternation of any cell, tissue or organ or groups of cells, tissues, or organs of plants including the root, leaf, shoot, fruit, petiole, trichome, flower, sepals, petals, hypocotyl, stigma, style, stamen, pollen, ovule, seed, embryo, endosperm, seed coat, aleurone, fibre nodule, cambium, wood, heartwood, parenchyma, sclerenchyma, seive element, phloem, or vascular tissue, amongst others.

In a preferred embodiment, over-expression of a cis-prenyltransferase protein or functional fragment thereof, operably associated with a DNA sequence regulating its expression, will result in genetically transformed plant cells having an altered growth phenotype wherein the growth rate to maturity is accelerated and/or yield is increased. An “increased” or “accelerated growth rate” will refer to either the total plant or the growth rate of specific tissues/organs of the plant (e.g., the rate of root or shoot growth, or the timing associated with commencement of flowering, seed set, or ripening of fruits). “Increased yield” refers to an increased or enhanced biomass of any harvestable material of the transgenic plant, (either the total plant or specific tissues/organs of the plant [e.g., root, leaf, shoot, fruit, petiole, trichome, flower, sepals, petal, hypocotyl, stigma, style, stamen, pollen, ovule, seed, bulb, embryo, endosperm, seed coat, aleurone, fibre, nodule, cambium, wood, heartwood, parenchyma, sclerenchyma, seive element, phloem, or vascular tissue]). Thus, increased yield includes, but is not limited to: increased or enhanced biomass of the root or shoot, seed production, grain yield, fruit size, nitrogen fixing capacity, nodule size, tuber formation, stem thickness, endosperm size, and number of fruit per plant, etc. Increased yield may also refer to accumulation of metabolites and/or the sink/source relationships in the total plant or specific portions of the plant.

Increased growth rate is another measure of the effect of the expression of the present cis-prenyltransferase in plants. As used herein “increased growth rate” will include but not be limited to characteristics selected from group consisting of: decreased time to germination, increased root growth rate, increased shoot growth rate, decreased time to flowering, decreased time for fruit maturation, and decreased time of seed setting.

As an example of the accelerated or increased growth rate to maturity and/or increased yield that can be observed by following the methodology of the present invention, in a preferred embodiment, the model organism Arabidopsis was transformed with a cis-prenyltransferase gene. The phenotype of the transformant plants included the following: decreased time to bolting and flowering, and increased seed yield at maturity. Additionally, significant modifications in plant architecture were observed, based on an increased leaf size and total leaf area, and increased plant height prior to maturity.

In view of the teachings herein, it will be appreciated by one of skill in the art that any desired plant species that supports production of a cis-prenyltransferase could be modified to exhibit a modified growth rate to maturity and/or yield and/or modifications in plant architecture. These broad modifications to overall plant growth phenotypes include—but are not limited to—the initiation, promotion, stimulation or enhancement of, or inhibition or diminishment of: cell division, seed development, tuber formation, shoot initiation, leaf initiation, root growth, properties of apical dominance, etc. Any transformed plant obtained according to the invention can be used in a conventional breeding scheme or in in vitro plant propagation to produce more transformed plants with the same characteristics and/or can be used to introduce the same characteristic in other varieties of the same or related species. The plants of this invention are fertile (i.e., capable of self- or cross-pollination with other plants of the same species to produce seed) and such plants are included as a part of the invention. Seeds obtained from the transformed plants genetically also contain the same characteristics and are capable of germination and growth. These seeds are also part of the invention herein.

In yet another aspect, the invention also relates to harvestable parts and to propagation material of the transgenic plants according to the invention containing transgenic plant cells over-expressing a cis-prenyltransferase. Harvestable parts can be in principle any useful parts of a plant (e.g., flowers, pollen, seedlings, tubers, leaves, stems, fruit, seeds, roots, etc.). Propagation material includes, but is not limited to: seeds, fruits, cuttings, seedlings, tubers, and rootstocks, etc.

Most advantageously to the purposes of the present invention, overexpression of cis-prenyltransfereases for the purposes of generating transformant plants (exhibiting modified characteristics of growth rate and/or yield and/or modifications in plant architecture) does not affect other plant properties in ways deleterious to agriculture, silviculture, horticulture, floriculture or cell culture.

### Pathway Engineering

As one skilled in the art will appreciate, it may be useful to manipulate the polyisoprenoid biosynthetic pathway of a plant as a mechanism for modifying the level of cis-prenyltransferase expression. Methods of manipulating genetic pathways are common and well known in the art. Selected genes in a particularly pathway may be unregulated or down-regulated by variety of methods. Additionally, competing pathways in an organism may be eliminated or sublimated by gene disruption and similar techniques.

Once a key genetic pathway has been identified and sequenced, specific genes may be up-regulated to increase the output of the pathway. For example, additional copies of the targeted genes may be introduced into the host cell on multicopy plasmids such as pBR322. Alternatively the target genes may be modified so as to be under the control of non-native promoters. Where it is desired that a pathway operate at a particular point in a cell cycle, regulated or inducible promoters may used to replace the native promoter of the target gene. Similarly, in some cases the native or endogenous promoter may be modified to increase gene expression. For example, endogenous promoters can be altered in vivo by mutation, deletion, and/or substitution (see, Kmiec, U.S. Pat. No. 5,565,350; Zarling et al., PCT/US93/03868).

Alternatively, it may be necessary to reduce or eliminate the expression of certain genes in the target pathway or in competing pathways that may serve as competing sinks for energy or carbon. Methods of down-regulating genes for this purpose have been explored.

For example, where sequence of the gene to be disrupted is known, one of the most effective methods for gene down-regulation is targeted gene disruption where foreign DNA is inserted into a structural gene so as to disrupt transcription. This can be effected by the creation of genetic cassettes comprising the DNA to be inserted (often a genetic marker) flanked by sequences having a high degree of homology to a portion of the gene to be disrupted. Introduction of the cassette into the host cell results in insertion of the foreign DNA into the structural gene via the native DNA replication mechanisms of the cell (see for example Hamilton et al. J. Bacteriol. 171:4617-4622 (1989); Balbas et al. Gene 136:211-213 (1993); Gueldener et al. Nucleic Acids Res. 24:2519-2524 (1996); and Smith et al. Methods Mol. Cell. Biol. 5:270-277 (1996)).

Alternative methods are available to reduce or eliminate expression of a specific gene of interest encoding a polypeptide, if desirable in plants for some applications. In order to accomplish this, a chimeric gene designed for co-suppression of the polypeptide can be constructed by linking a gene or gene fragment encoding that polypeptide to plant promoter sequences. Antisense technology requires that a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA which encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan. Nonetheless, either the co-suppression or antisense chimeric genes could be introduced into plants via transformation wherein expression of the corresponding endogenous genes is reduced or eliminated.

Finally, one recent variation upon “classical” antisense and cosuppression methodologies is embodied in WO 02/00904, published on Jan. 3, 2002. Specifically, it was found that suitable nucleic acid sequences and their reverse complement can be used to alter the expression of any mRNA encoding a protein of interest which is in proximity to the suitable nucleic acid sequence and its reverse complement. Surprisingly, the suitable nucleic acid sequence and its reverse complement can be either unrelated to any endogenous RNA in the host or can be encoded by any nucleic acid sequence in the genome of the host provided that the nucleic acid sequence does not encode any target mRNA or any sequence that is substantially similar to the target mRNA. A preferred artificial and non-naturally occurring, sequence is that encoded by the peptide “ELVISLIVES” (SEQ ID NO:33). This approach permits a very efficient and robust approach to achieving single, or multiple, gene co-suppression using single plasmid transformation.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression or similar methodologies thereto (U.S. Pat. No. 5,190,931; U.S. Pat. No. 5,107,065; U.S. Pat. No. 5,283,323; WO 02/00904). An antisense or cosuppression construct would act as a dominant negative regulator of gene activity. While conventional mutations can yield negative regulation of gene activity, these effects are most likely recessive. The dominant negative regulation available with a transgenic approach may be advantageous from a breeding perspective. In addition, the ability to restrict the expression of a specific phenotype to the reproductive tissues of the plant by the use of tissue-specific promoters may confer agronomic advantages relative to conventional mutations that may have an effect in all tissues in which a mutant gene is ordinarily expressed.

A person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds, and is not an inherent part of the invention. For example, one can screen by looking for changes in gene expression by using antibodies specific for the protein encoded by the gene being suppressed, or one could establish assays that specifically measure enzyme activity. A preferred method will be one that allows large numbers of samples to be processed rapidly, since it will be expected that a large number of transformants will be negative for the desired phenotype.

Although targeted gene disruption and antisense technology offer effective means of down-regulating genes where the sequence is known, other less specific methodologies have been developed that are not sequence based. For example, cells may be exposed to UV radiation and then screened for the desired phenotype. Mutagenesis with chemical agents is also effective for generating mutants and commonly used substances include chemicals that affect nonreplicating DNA such as HNO₂ and NH₂OH, as well as agents that affect replicating DNA such as acridine dyes, notable for causing frameshift mutations. Specific methods for creating mutants using radiation or chemical agents are well documented in the art. See, for example: Thomas D. Brock in Biotechnology: A Textbook of Industrial Microbiology, 2^{nd} ed., Brock, T. D., Ed.; Sinauer Associates: Sunderland, Mass., 1989; or Deshpande, Mukund V., Appl. Biochem. Biotechnol., 36: 227 (1992).

Another non-specific method of gene disruption is the use of transposable elements or transposons. Transposons are genetic elements that insert randomly in DNA but can be later retrieved on the basis of sequence to determine where the insertion has occurred. Both in vivo and in vitro transposition methods are known. Both methods involve the use of a transposable element in combination with a transposase enzyme. When the transposable element or transposon is contacted with a nucleic acid fragment in the presence of the transposase, the transposable element will randomly insert into the nucleic acid fragment. The technique is useful for random mutagenesis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass., based upon the bacterial transposon Tn7; and the EZ::TN Transposon Insertion Systems, available from Epicentre Technologies, Madison, Wis., based upon the Tn5 bacterial transposable element).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

Standard recombinant DNA and molecular cloning techniques used herein are well known in the art and are described by Sambrook et al., Molecular Cloning: A Laboratory Manual, 2^{nd} ed., Cold Spring Harbor Laboratory: Cold Spring Harbor, N.Y., 1989 (hereinafter “Maniatus”); and by T. J. Silhavy, M. L. Bennan, and L. W. Enquist, Experiments with Gene Fusions, Cold Spring Harbor Laboratory: Cold Spring Harbor, N.Y., 1984; and by Ausubel et al., Current Protocols in Molecular Biology, published by Greene Publishing Assoc. and Wiley-Interscience, 1987.

Nucleotide and amino acid percent identity and similarity comparisons were made using the BLAST (Basic Local Alignment Search Tool; Altschul et al., J. Mol. Biol. 215:403-410 (1993); see also www.ncbi.nlm.nih.gov/BLAST/) algorithms and also the Vector NTI suite of programs, applying default parameters unless indicated otherwise.

The meaning of abbreviations is as follows: “sec” means second(s), “min” means minute(s), “hr” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole, “g” means gram(s), “μg” means microgram(s), “ng” means nanogram(s), “U” means unit(s), “bp” means base pair(s), and “kB” means kilobase(s).

### Example 1

### Bioinformatic Analysis of Publicly Available Cis-Prenyltransferase Homologs

Arabidopsis is well-known as a “model organism” in plant science for investigations in a wide range of processes involved in controlling growth and development of flowering plants. This is largely based on: 1.) its small genome (five chromosomes and approximately 25,000 genes); 2.) rapid life cycle (about 6 weeks from seed to seed); 3.) prolific seed production; 4.) small size, thereby allowing a large number of plants to be grown in limited space; and 5.) ability to extrapolate results obtained in Arabidopsis to other agriculturally important crops. For these reasons, Arabidopsis was selected as a model organism in the present study, in which it would be desirable to express various cis-prenyltransferase genes.

Example 1 describes the selection of three exogenous cis-prenyltransferase homologs from rubber tree, soybean, and rice for expression in Arabidposis. Additionally, an endogenous gene (Apt5) of Arabidopsis having significant homology to other known cis-prenyltransferase genes was identified and selected for over-expression.

### Identification of Exogenous Cis-Prenyltransferase Homologs

Previous work (U.S. Pat. No. 6,645,747) has described the preparation of cDNA libraries from Hevea brasiliensis, Oryza sativa, and Glycine max and the identification of cis-prenyltransferase homologs from these libraries. Three of these homologs are summarized below in Table 2, according to their original clone name and designated gene name.

TABLE 2
cDNAs Identified as cis-Prenyltransferase Homologs

| | | SEQ ID | Gene |
|---|---|---|---|
| Clone | Source | NO | name |
| ehb2c.pk001.d17 | Hevea brasiliensis | 1 | Hpt2 |
| rr1.pk0050.h8 | Oryza sativa | 3 | Rpt1 |
| sl1.pk0128.h7 | Glycine max | 5 | Spt1 |

Comparison of the nucleotide sequences of the rubber, soybean, and rice ESTs (SEQ ID NO:1, SEQ ID NO:3, SEQ ID NO:5) with those of representative bacterial, yeast, and Arabidopsis cis-prenyltransferases confirmed that each of these homologs exhibited significant homology with known examples of the cis-prenyltransferase gene family. Specifically, SEQ ID NO:1, SEQ ID NO:3, SEQ ID NO:5 were examined against Micrococcus luteus UPPS (Shimizu, N., et al., J. Biol. Chem. 273:19476-19481 (1998); GenBank Accession No. AB004319), yeast rer2 (Sato, M., et al., Mol. Cell. Biol. 19, 471-483 (1999); AB013497), yeast srt1 (AB013498), and Arabidopsis Apt1 (Oh, S. K., et al., J. Biol. Chem. 275:18482-18488 (2000); Cunillera, N., et al., FEBS Letts. 477:170-174 (2000); AF162441).

### Identification of Endogenous Cis-Prenyltransferase Homologs

In addition to the cis-prenyltransferase genes identified above, several Arabidopsis thaliana genomic DNA fragments containing putative cis-prenyl transferase gene sequences were identified in public databases. Specifically, publicly available sequences of bacterial and yeast cis-prenyl transferases were used to conduct BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL and DDBJ databases) using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. Nature Genetics 3:266-272 (1993)) provided by the NCBI.

Putative cis-prenyl transferase gene sequences from Arabidopsis thaliana were additionally identified by a number of methods, including the following: 1) keyword searches (e.g., “undecaprenyl”), 2) searches of the database using the TBLASTN algorithm provided by the National Center for Biotechnology Information (NCBI) and short fragments of conserved sequence present in known cis-prenyltransferases (conserved domains I-V, as described by Apfel et al., J. Bacterol. 81:483-492 (1999)). These sections of conserved sequence were expected to be diagnostic for the cis-prenyltransferase family of enzymes.

One gene, designated Apt5, from Arabidopsis thaliana chromosome 5 genomic DNA (GenBank Accession Number AB011483), contains an 813 bp open reading frame (SEQ ID NO:20) with no intron sequences, and encodes a protein with 271 amino acids (SEQ ID NO:21). This protein has extensive homology to known microbial and plant cis-prenyltransferase sequences. It was decided to include this gene in the present Arabidopsis transformation experiments to determine the effect of overexpression of an endogenous gene.

### Comparison of Cis-Prenyltransferase Homologs

A more informative comparison of deduced amino acid sequences of the ORFs encoded by these cDNAs was carried out using the Vector NTI AlignX program, which uses the ClustalW algorithm to align and compare sequences for similarity and identity. A comparison of the Hpt2, Rpt1, Spt1, and Apt5 deduced amino acid sequences with that of Arabidopsis Apt1 (SEQ ID NO:32; GenBank Accession Number AF162441) is presented in Table 3.

TABLE 3
Identity Comparison Using the ClustalW Program of the Deduced
Amino Acid Sequences from Plant cis-Prenyltransferases with
the Arabidopsis Apt1 cis-Prenyltransferase

| | | % similarity | % identity |
|---|---|---|---|
| | SEQ ID | with Arabidopsis | with Arabidopsis |
| Gene | NO | homolog Apt1 | homolog Apt1 |
| Hpt2 | 2 | 36.9 | 22.9 |
| Rpt1 | 4 | 36.8 | 23.4 |
| Spt1 | 6 | 54.8 | 40.9 |

It is clear from this analysis that these sequences (exogenous to Arabidopsis) encode polypeptides with significant similarity to a known cis-prenyltransferase.

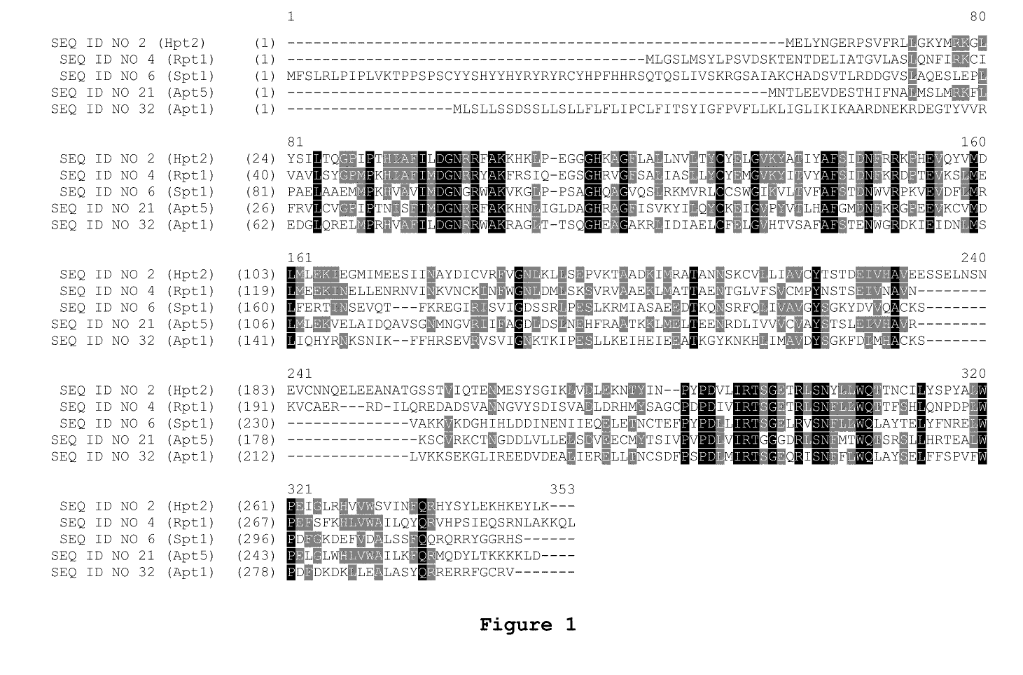

Furthermore, alignment of the deduced amino acid sequence of these cDNAs encoding Hpt2, Rpt1, and Spt1 (exogenous to Arabidopsis). and Apt5 (endogenous to Arabidopsis) with a known plant cis-prenyltransferase Apt1 (FIG. 1) using the CLUSTALW program within the VECTOR NTI suite of programs reveals the presence of the conserved domains characteristic of this gene family (see, Apfel et al., J. Bacteriol. 81:483-492 (1999)).

### Example 2

### Construction of Cis-Prenyltransferases Expression Vectors

The present Example describes construction of a binary vector for expression of the cis-prenyltransferase genes identified in Example 1. Each cis-prenyltransferase was amplified by PCR and cloned into an appropriate vector for subsequent expression in Arabidopsis.

Construction of pBI-35S

A binary vector, pBI-35S, was constructed for expression of several cis-prenyltransferase genes by ligating an 800 bp Hind III-Xba I CaMV35 promoter DNA fragment (Guilley H, et al., Cell 30(3):763-73 (1982)) into the corresponding sites of the vector pBIB/NPT (Detlef Becker, Nucleic Acids Research 18(1):203 (1990)) to yield the binary vector pBI-35S.

Construction of pGV827

Plasmid pGV827 contains the GFP gene under the control of the ³⁵S cauliflower mosaic virus promoter and the nopaline synthase 3′ translation termination sequence. It is derived from the commercially purchased vector pBIN19 (CloneTech; Frisch, R. A. et al., Plant Molecular Biology 27:405-409 (1995)) and from psmGFP (GenBank Accession Number U70495; Davis S. J., and R. D. Vierstra. Plant Mol Biol 36(4):521-8 (1998)). Specifically, psmGFP was digested with EcoRI and HindIII, to release the fragment containing 35S::GFP::nos. This was then ligated into EcoRI- and HindIII/-digested pBIN19 to create pGV827.

### Amplification and Cloning of Cis-Prenyltransferases

Chimeric genes comprising Hevea, rice and soybean cis-prenyltransferases (SEQ ID NO: 1, SEQ ID NO:3, SEQ ID NO:5) in sense orientation were constructed by polymerase chain reaction (PCR) from plasmids containing the Hevea, rice or soybean cis-prenyltransferase homologs, for expression in Arabidopsis thaliana. In contrast, the Apt5 gene (SEQ ID NO:20) was cloned by PCR amplification using Arabidopsis thaliana genomic DNA as a template.

More specifically, Hpt2 was amplified from clone ehb2c.pk001.d17, using oligonucleotide primers HW8 (SEQ ID NO:14) and HW12 (SEQ ID NO:15). The amplified Hpt2 cDNAs were digested with XbaI and KpnI and separated on an agarose gel. The DNA fragment was isolated and purified using a QIAquick Gel Extraction Kit, according to the manufacturer's instructions (Qiagen Inc., Chatsworth, Calif.). The purified DNA fragment was cloned into the corresponding sites of the binary vector pBI-35S (supra) to yield 35S::Hpt2.

Apt5 was isolated from A. thaliana genomic DNA, using primers Apt5/XbaI (SEQ ID NO:22) and Apt5/KpnI (SEQ ID NO:23). These primers were designed to include specific restriction sites at each end to facilitate in cloning. The amplified Apt5 gene was digested with XbaI and KpnI and separated on an agarose gel. The DNA fragment, ca. 850 bp in length, was isolated and purified using a QIAquick Gel Extraction Kit (Qiagen Inc., Chatsworth, Calif.); subsequently, the purified DNA fragments were cloned into a pBluescript vector according to manufacturer's instructions (Stratagene, LaJolla, Calif.). The Xba I-Kpn I DNA fragment encoding the Apt5 gene (SEQ ID NO:20) was then cloned into the pBI-35S vector, yielding the construct 35S::Apt5.

Rpt1 and Spt1 were isolated in a manner similar to that for Hpt2; however, BamHI and SacI cloning sites were incorporated into the oligonucleotide primers to provide proper orientation of the DNA fragment upon insertion into the binary vector pGV827. Specifically:
Rpt1 was amplified from clone rr1.pk0050.h8 using primers JK1 (SEQ ID NO:16) and JK7 (SEQ ID NO:20); and
Spt1 was amplified from clone sl1.pk0128.h7 using primers JK3 (SEQ ID NO:18) and JK4 (SEQ ID NO:19).
   PCR products were cloned into the pGEM T-easy vector using a TA-cloning kit (Promega Corporation, Madison, Wis.). Subsequently, the plasmids were digested with BamHI and SacI and the cDNA fragments encoding Rpt1 and Spt1 were isolated by agarose gel purification. The fragments were ligated into pGV827, to yield the resulting rice and soybean gene expression constructs 35S::rr1 and 35S::sl1, respectively. Expression vectors 35S::Hpt2, 35S::rr1, 35S::sl1, and 35S::Apt5 were each individually transformed into E. coli. To verify integrity of the amplified DNAs, plasmids were isolated and purified using QIAFilter cartridges (Qiagen Inc., Chatsworth, Calif.) according to the manufacturer's instructions. Sequence was generated on an ABI Automatic sequencer using dye terminator technology (U.S. Pat. No. 5,366,860; EP 272007) and using a combination of vector-specific primers. Sequence editing was performed in Vector NTI.

### Example 3

### Transformation of Expression Vectors Containing Cis-Prenyl Transferases into Arabidopsis thaliana

The present Example describes the transformation of plasmids 35S::Hpt2, 35S::Apt5, 35S::rr1 and 35S::sl1 (from Example 2) into Arabidopsis.

Specifically, plasmids 35S::Hpt2 and 35S::Apt5 were transformed into Agrobacterium tumefaciens strain C58 using a freeze-thaw method (Holsters et al., Mol. Gen. Genet. 163:181-187 (1978)). Arabidopsis thaliana plants were transformed via Agrobacterium-mediated transformation (Clough S. J., Bent A. F.; Plant Journal 16(6): 735-43 (1998)).

Plasmids 35S::rr1 and 35S::sl1 were also transformed into the Agrobacterium tumefaciens strain C58C1 by a freeze-thaw method (Holsters et al., supra). However, Agrobacterium lines bearing the binary vector constructs were selected using PCR and used to transform Arabidopsis thaliana using the floral dip method (Clough S. J., Bent A. F.; supra).

### Example 4

### Expression of Cis-prenyltransferase Genes in Arabidopsis thaliana

Example 4 describes the growth and identification of 4 lines (Rpt1 8-1, Spt1 10-4, Hpt2 16-2 and Apt5 1-4) of transgenic plants, carrying plasmids 35S::rr1, 35S::sl1, 35S::Hpt2, and 35S::Apt5, respectively. Reverse-transcriptase PCR was performed to confirm transgene expression.

### Growth and Identification of Transformant Lines of Arabidopsis

The seeds produced from infected plants transformed with vectors 35S::Hpt2, 35S::rr1, 35S::sl1, and 35S::Apt5 were germinated on agar plates containing 100 μg/mL kanamycin. Arabidopsis plants resistant to kanamycin were selected and planted into soil. Seed was collected from these plants, and germinated on agar plates containing 100 μg/mL kanamycin. Three 35S::Rpt1 lines, five 35S::Spt1 lines, five 35S::Hpt2 and three 35S::Apt5 lines were selected as segregating 3:1 for resistance after germination on agar plates containing 100 μg/mL kanamycin. Subsequent selection for 100% resistance yielded three 35S::Rpt1 lines, three 35S::Spt1 lines, four 35S::Hpt2 and two 35S::Apt5 lines. Of these, four were selected for further study and designated as Rpt1 8-1, Spt1 10-4, Hpt2 16-2 and Apt5 1-4, respectively.

### Analysis of Transgene Expression by RT-PCR

Relative quantitative reverse-transcriptase PCR(RT-PCR) was performed to confirm transgene expression and to compare expression levels among the four lines, using primers specific to the cis-prenyltransferase sequences. RNA was prepared from Arabidopsis leaves of the following lines: Apt5 1-4, Hpt2 16-2, Rpt1 8-1-5-4 and Spt1 10-4-3-3, using the RNAeasy Midi-Kit (Qiagen, Valencia, Calif.), according to the manufacturer's supplied protocol for samples from plant tissue. RNA was quantified on a fluorometer (Turner Designs, Sunnyvale, Calif.).

To control for variations in RNA quality, quantitation errors and random variation in RT-PCR, “multiplex” RT-PCR was performed. Multiplex PCR utilizes two or more primer sets in one reaction: one set to amplify the cDNA of interest and one set to amplify an invariant endogenous control. Primers used for amplification of each of the specific cDNAs were:
Apt5s (SEQ ID NO:24) and Apt5as (SEQ ID NO:25) for Apt5;
H2s (SEQ ID NO:26) and H2as (SEQ ID NO:27) for Hpt2;
NKH33 (SEQ ID NO:28) and NKH34 (SEQ ID NO:29) for Rpt1; and,
NKH35 (SEQ ID NO:30) and NKH36 (SEQ ID NO:31) for Spt1.
   The primer sets used to amplify the endogenous control were the 18S PCR primer set and 18S PCR competimer set, supplied in the QuantumRNA Plant 18S Internal Standard Kit (Ambion, Austin, Tex.). One-step RT-PCR was performed on 1 ng and 2 ng of each RNA sample, according to manufacturer's supplied protocol (Qiagen). Amplification was carried out as follows: initial incubation at 50° C. for 30 min; initial denaturation at 95° C. for 15 min, followed by 28 cycles of 94° C. (1 min), 52° C. (1 min) and 72° C. (1.5 min). A final extension cycle of 72° C. for 10 min was performed.

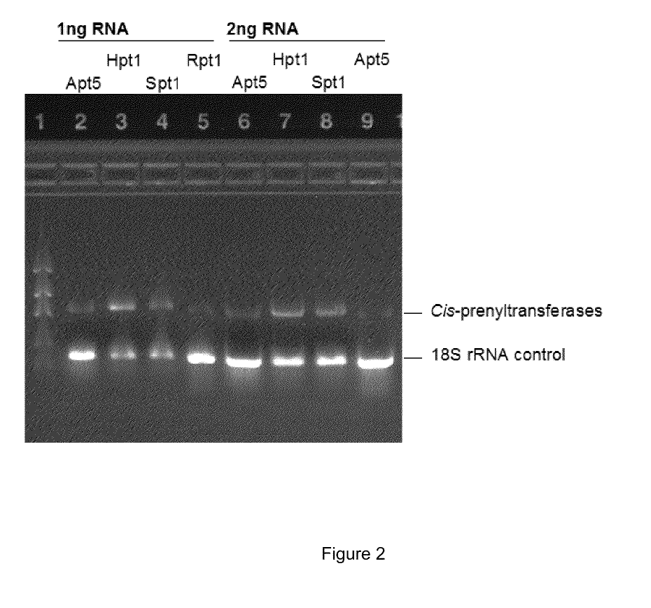

The RT-PCR results of cis-prenyltransferase transcripts from transgenic Arabidopsis plants are shown in FIG. 2 and indicated the expression of the cis-prenyltransferase transgenes in each of the lines tested. Relative transgene expression levels from lowest to highest were: Rpt1 8-1-5-4, Apt5 1-4, Spt1 10-4-3-3, Hpt2 16-2. These transgenic lines were used in all subsequent experiments, where they are designated by the abbreviations Rpt1, Apt5, Spt1 and Hpt2.

### Example 5

### Analysis of Arabidopsis Transgenic for Cis-Prenyltransferases

Example 5 shows that constitutive expression of individual cis-prenyltransferase genes (expressed from plasmids 35S::Hpt2, 35S::Apt5, 35S::rr1 and 35S::sl1) is sufficient to modify growth, architecture and yield in transgenic Arabidopsis plants. In the majority of cases, constitutive expression of individual cis-prenyltransferase genes resulted in an enhanced rate of growth to maturity and considerably increased seed yield, characteristics of central importance to the commercial uses of plants.

### Growth Conditions

Seed obtained from lines homozygous for resistance to kanamycin (Rpt1 8-1-5, Spt1 10-4-3, Hpt2 16-2 and Apt5 14; Example 4) were sown in Metro-Mix and grown in either constant light or with a 12 hr photoperiod (fluorescent supplemented with incandescent) at 22° C., 50% relative humidity. Plants were watered with a nutrient solution containing 1 mM ammonium phosphate, 1 mM potassium nitrate, 1 mM calcium nitrate, 2 mM magnesium sulfate, 1 mM ammonium nitrate, 5 ppb Fe and the following trace elements: manganese, chloride, boric acid, zinc sulfate, cupric sulfate, and molybdic acid. Plants (8-10 individual plants from each transgenic line) were observed during growth to determine whether the transgenes, overexpressed using the 35S promoter, affected growth rate. Overall, it was observed that most plants constitutively expressing cis-prenyltransferase transgenes exhibited accelerated growth to maturity as compared to untransformed (wild-type) plants.

Modified Growth Rate and/or Yield for Leaves

Marked effects of the transgenes on leaf dimensions were observed, with the transgenic plants exhibiting on average longer leaves (Tables 4 and 5) and broader leaves (Tables 6 and 7). Leaf length was measured as leaf rosette radii; leaf width was measured at widest point.

TABLE 4
Effect of cis-prenyltransferase transgene expression
on leaf rosette radius (grown under constant light)

| Overexpressed | Rosette radius (cm) at | Rosette radius (cm) at 36 |
|---|---|---|
| transgene | 30 dps¹ (std. dev.) | dps¹ (std. dev.) |
| None (wild-type) | 2.09 (0.47) | 2.4 (0.31) |
| Rpt1 | 2.93 (0.65) | 3.13 (0.57) |
| Apt5 | 2.91 (0.4) | 3.11 (0.35) |
| Spt1 | 3.23 (0.21) | 3.33 (0.71) |
| Hpt2 | 3.17 (0.75) | 3.23 (0.25) |

¹Days post sowing

TABLE 5
Effect of cis-prenyltransferase transgene expression on
leaf rosette radius (grown with a 12 hr photoperiod)

| | Rosette radius (cm) at |
|---|---|
| Overexpressed transgene | 28 dps¹ (std. dev.) |
| None (wild type) | 2.30 (0.94) |
| Rpt1 | 3.46 (1.23) |
| Apt5 | 4.89 (0.77) |
| Spt1 | 4.82 (0.62)) |
| Hpt2 | 4.88 (1.39) |

¹Days post sowing

TABLE 6
Effect of cis-prenyltransferase transgene expression
on leaf width (grown under constant light)

| Overexpressed | Leaf width (mm) at | Leaf width (mm) at |
|---|---|---|
| transgene | 21 dps¹ (std. dev.) | 36 dps¹ (std. dev.) |
| None (wild-type) | 5.38 (1.38) | 11.38 (1.86) |
| Rpt1 | 5.8 (1.54) | 13.15 (3.62) |
| Apt5 | 7.45 (1.57) | 18.35 (3.20) |
| Spt1 | 8.25 (1.58) | 16.12 (2.99) |
| Hpt2 | 8.0 (0.75) | 19.5 (4.36) |

¹Days post sowing

TABLE 7
Effect of cis-prenyltransferase transgene expression
on leaf width (growth with a 12 hr photoperiod)

| | Leaf width (mm) at |
|---|---|
| Overexpressed transgene | 28 dps¹ (std. dev.) |
| None (wild type) | 8.16 (2.72) |
| Rpt1 | 10.81 (3.00) |
| Apt5 | 17.23 (2.05) |
| Spt1 | 18.14 (4.50) |
| Hpt2 | 17.78 (4.75) |

¹Days post sowing

Both of these parameters were increased ca. 1.5-fold in plants expressing the soybean and Hevea cis-prenyltransferases, for example. Overexpression of the rice Rpt1 gene also resulted in longer leaves, although in this case their average width was indistinguishable from wild-type plants.
Modified Growth Rate and/or Yield for Plant Height

Marked effects of transgene expression were also observed on plant height, when compared at fixed times after sowing (Table 8).

TABLE 8
Effect of cis-prenyltransferase transgene expression
on plant height (grown under constant light)

| Overexpressed | Height (cm) at | Height (cm) at |
|---|---|---|
| transgene | 28 dps¹ (std. dev.) | 32 dps¹ (std. dev.) |
| None (wild-type) | 6.13 (5.47) | 15.36 (8.07) |
| Rpt1 | 4.2 (4.69) | 12.6 (13.3) |
| Apt5 | 16.56 (10.93) | 29.98 (11.67) |
| Spt1 | 25.79 (6.09) | 34.33 (6.28) |
| Hpt2 | 21.75 (9.9) | 33.31 (8.3) |

¹Days post sowing

Data shows that bolting plants expressing Apt5, Spt1 or Hpt2 were (on average) 3-3.5 times taller than wild-type plants 28 days after sowing. This effect was not observed in plants expressing Rpt1, which exhibited considerable variability for this measurement and whose average height was statistically indistinguishable from wild-type plants at both times measurements were made.

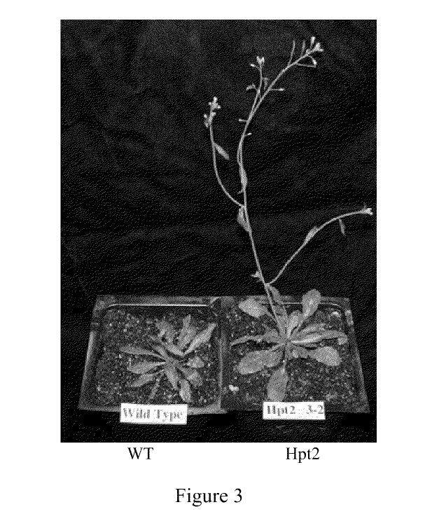

FIGS. 3, 4, and 5 clearly illustrate that plants constitutively expressing cis-prenyltransferases displayed a modified growth rate, as compared to wild type plants. Specifically, FIG. 3 is a comparison between transgenic Arabidopsis expressing the Hevea brasiliensis Hpt2 cis-prenyltransferase (line Hpt2 3-2) and a wild-type plant. Both plants were photographed 35 days after sowing and are representative examples of a population of plants. Clearly, overexpression of the Hpt2 cis-prenyltransferase resulted in increased plant growth, such that the genetically transformed plant reached maturity faster than the wild-type plant.

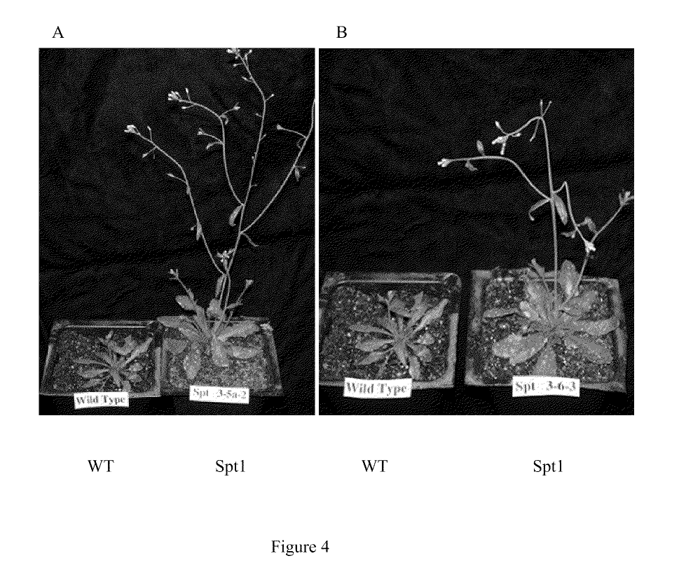

FIGS. 4A and 4B are comparisons between transgenic Arabidopsis expressing the Glycine max Spt1 cis-prenyltransferases (lines Spt2 3-5a-2 and Spt2 3-6-3, respectively) and wild-type plants. Again, plants were photographed 35 days after sowing and are representative examples of a population of plants. Expression of the Spt1 cis-prenyltransferase also resulted in accelerated growth of the transformed plant, relative to the wild-type, as determined by plant height. And, although not shown, over-expression of the endogenous Apt5 cis-prenyltransferase also led to a dramatic increase in the rate of growth to maturity of the transgenic plant, relative to the wild-type.

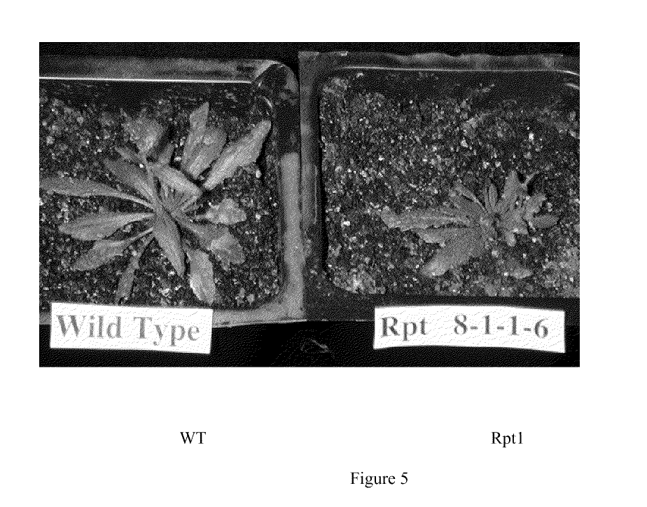

FIG. 5 is a comparison between transgenic Arabidopsis expressing the Oryza sativa Rpt1 cis-prenyltransferase (line Rpt 8-1-1-6) and a wild-type plant. Both plants were photographed 35 days after sowing and are representative examples of a population of plants. Rpt1 cis-prenyltransferase over-expression appeared to modify growth of Arabidopsis, leading to a somewhat reduced growth rate as compared to wild type.

Modified Growth Rate and/or Yield for Flowering and Seeds

The effect of the transgenes on flowering time and seed yield were also monitored (Table 9). The data shows that plants overexpressing Apt5, Hpt2 and Spt1 on average bolted earlier than wild-type plants and developed more numerous inflorescences. The most dramatic effect of transgene expression, however, was on final seed yield.

Seed yield was determined according to the average weight of seed per plant, since individual seed weight was not affected by expression of any of the transgenes. More specifically, 1000 seeds from either the wild-type or the transgenics plants (grown under constant light or with a 12 hr photoperiod) always weighed ca. 30 mg.

The data show that plants overexpressing cis-prenyltransferases produce more numerous seed than untransformed, wild-type plants. Those overexpressing Apt5, Hpt2 and Spt1 are capable of yielding 10-fold the number of seed that can be routinely obtained from wild-type Arabidopsis.

TABLE 9
Effect of cis-prenyltransferase transgene expression on bolting,
flowering and seed yield (grown under constant light)

| | | | | Seed yield |
|---|---|---|---|---|
| | | | No. | (average |
| Over- | Bolting time | No. leaves | inflores- | weight of |
| expressed | in dps¹ | at bolting | cences | seed per |
| transgene | (std. dev.) | (std. dev.) | (std. dev.) | plant) (mg) |
| None | 28.88 (2.80) | 14.11 (2.26) | 4 (0.866) | 23.8 |
| (wild-type) | | | | |
| Rpt1 | 30.37 (4.59) | 14.9 (3.03) | 5.8 (1.61) | 68.9 |
| Apt5 | 26.6 (3.53) | 13 (2.30) | 4.9 (0.99) | 187.52 |
| Spt1 | 25.25 (1.75) | 12.5 (0.92) | 4.5 (1.19) | 241.09 |
| Hpt2 | 25.75 (2.05) | 13.75 (1.90) | 4.38 (1.06) | 238.8 |

¹Days post sowing

TABLE 10
Effect of cis-prenyltransferase transgene expression
on seed yield (growth with a 12 hr photoperiod)

| | Seed yield |
|---|---|
| Overexpressed | (average weight of |
| transgene | seed per plant) (mg) |
| None | 36.9 |
| (wild-type) | |
| Rpt1 | 52.7 |
| Apt5 | 231.9 |
| Spt1 | 237.2 |
| Hpt2 | 240.5 |

### Example 6

### Further Analysis of Arabidopsis Transgenic for Cis-Prenyltransferases

Example 6 describes a more detailed examination of various characteristics of the transgenic Arabidopsis, expressing the Apt5, Hpt2, Spt1, and Rpt1 cis-prenyltransferases. As demonstrated in Example 5, constitutive expression of the cis-prenyltransferase genes is sufficient to modify growth, architecture and yield in transgenic Arabidopsis plants.

Seed from the parental wild-type Columbia and from 4 T3 lines of homozygous transformed Arabidopsis (Spt1 (event 10-4-3-3), Rpt1 (event 8-1-5-4), Hpt2 (event 16-2), Apt5 (event 14)) were obtained. Approximately 20 seed from each line were suspended in 1 mL of water and were put in the refrigerator for 2 d. Then, the seeds were planted on prewetted Metro Mix in 4-inch pots by pipetting the seed and water solution over 4 pots per genotype. The pots were then placed in a reach-in growth chamber and grown at 21° C. under continuous light until maturity.

After germination, the pots were thinned to 3 plants per pot, and thinned again to 1 per pot when a single healthy plant was observed in each pot. The plants were monitored for growth conditions daily and plant growth data and digital images were recorded at significant time points throughout each plant's life cycle (wherein plants were at their maximum size at 34 d after planting). Specifically, the following observations were made (all results represent those of the genotype average, unless specified to be otherwise):
Days to germination (recorded as the day when 90% of the plants had exposed cotyledons);
Days to first flowering;
Number of leaves at 26 days;
Average leaf size and total leaf area (wherein leaf measurements from 10 to 12 leaves from each genotype were taken by using digital image analysis (recorded as pixels) and averaged to get a genotype average);
Stem diameter at 34 days (measured at the base of each plant and averaged for each genotype);
Stem length or plant height at 34 days;
Number of siliques on the mainstem at 34 days;
Average silique length;
Number of bolts or stems per plant at 34 days;
Days until first mature seed;
Average number of seeds per silique (determined by counting each seed from 3 average looking siliques from representative plants and averaged);
Number of siliques per plant at plant maturity (recorded from a representative sample of plants from each genotype);
Total number of seed per plant (generated from the number of siliques per plant and the number of seeds per silique); and
Total seed weight (determined after harvesting all of the seed from each plant and reported as an average of all of the plants from each genotype).
   The results of these observations are summarized in Table 11 below.

TABLE 11
Effect of cis-prenyltransferase transgene expression in Arabidopsis

| | | | | Avg. | Total | | | |
|---|---|---|---|---|---|---|---|---|
| Over- | Days | Days | | leaf | leaf | Stem | Stem | # Siliques |
| expressed | to | to 1st | # | size | area | diameter | length | per |
| transgene | germination | flower | Leaves | (pixels) | (pixels) | (mm) | (mm) | mainstem |
| None | 7 | 27.5 | 8 | 263 | 2104 | 1 | 210 | 11 |
| (wild-type) | | | | | | | | |
| Rpt1 | 6 | 26 | 9.5 | 540 | 5130 | 1.8 | 292 | 15 |
| Apt5 | 6 | 26 | 8.5 | 772 | 6562 | 1.8 | 400 | 20 |
| Hpt2 | 5 | 23 | 8.5 | 1122 | 9537 | 1.5 | 432 | 30 |
| Spt1 | 5 | 23 | 8.75 | 1079 | 9441.3 | 2 | 435 | 32 |

| | | | Days to | | # | | Avg. | |
|---|---|---|---|---|---|---|---|---|
| Over- | Silique | | first | # | Siliques | Avg. | Total | Days to |
| expressed | length | # | mature | Seed/ | at | Total # | seed | seed |
| transgene | (mm) | Bolts | seed | silique | maturity | seed | wgt (g) | harvest |
| None | 9 | 1 | 42 | 16 | 129 | 2,110 | 0.016 | 50 |
| (wild-type) | | | | | | | | |
| Rpt1 | 16 | 5 | 38 | 28 | 131 | 4,164 | 0.043 | 50 |
| Apt5 | 18 | 5 | 38 | 53 | 124 | 6,919 | 0.102 | 50 |
| Hpt2 | 19 | 5 | 37 | 64 | 383 | 24,319 | 0.266 | 50 |
| Spt1 | 20 | 5 | 37 | 67 | 434 | 29,134 | 0.302 | 50 |

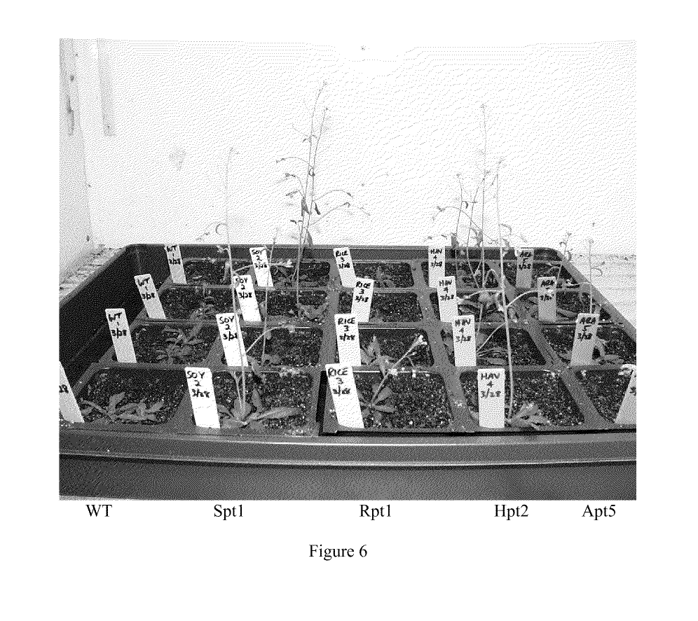

From the results presented above and the representative results shown in FIG. 6 (taken at 26 d after planting), the increased plant growth rates in most of the transgenic lines were very obvious. The following general conclusions concerning the modified growth rates and/or yield and/or plant architectures observed can be summarized as shown below:
Time to germination varied somewhat between transgenic lines, but all of the lines emerged 1 to 2 days (10-25%) earlier than the wild-type. The Spt1 and Hpt2 genotypes germinated most rapidly than the Apt5 and Rpt1 genotypes.
The length of time until the first flower appeared on each transgenic genotype was approximately 15% earlier than the wild-type variety. Flowering was observed on the Rpt1 and Apt5 lines 1 to 2 days earlier than the wild-type, while the Hpt2 and Spt1 lines were 4 to 5 days earlier.
The number of leaves present in the rosette was not significantly different from that of wild-type. However, the individual leaf size and the total leaf area of the plant was dramatically increased by up to 4 fold in the transgenic lines versus the wild-type. More specifically, expression of the Hpt2 and Spt1 cis-prenyltransferases had the greatest effect on leaf architecture, followed by Apt5 and Rpt1.
The stem diameters for each of the transgenic lines were significantly greater than the wild-type.
Concerning stem length, number of siliques per mainstem, average silique length, and the number of bold per plant, each of the 4 transgenic lines exhibited significantly increased growth and/or yield relative to the wild-type. Specifically, the transgenic lines were 2 times taller, the number and length of the siliques was 2-3 fold bigger, and the number of bolts was 5 times greater.
The transgenic lines began producing mature seed about 12% faster than the wild-type. Seed yields from the cis-prenyltransferase-expressing plants were 5 to 10 times greater than wild-type yields. The increased number of siliques and increased size of the siliques contributed to the large increase in yields. As demonstrated in Table 11, the Spt1 and Hpt2 lines yielded the most seeds, followed by the Apt5 and Rpt1 lines.
Despite the differences observed between the transgenic plants and the wild-type plants concerning rate of growth and/or yield and/or plant architecture, the time to seed harvest was still about the same for both transgenics and wild-type (50 days).
   In summary, the data clearly shows that over-expression of the cis-prenyltransferase genes in Arabidopsis results in dramatic modification to the growth rates and/or yield and/or architecture of the transgenic plants, as compared to the wild-type. Seed yields are greatly increased due to the increased number and size of the siliques in the Hevea (Hpt2) and Soy (Spt1) lines. Transgenic plant size and leaf area were also significantly increased, which could translate into more biomass per pot (or acre). This could be an especially beneficial result in any plant species that is grown commercially for biomass production (e.g., sugarcase, tobacco, cotton, etc.).

### Example 7

### Analysis of Arabidopsis Transgenic for Cis-Prenyltransferases

Example 7 again shows that constitutive expression of individual cis-prenyltransferase genes (expressed from plasmids 35S::Hpt2, 35S::Apt5, 35S::rr1 and 35S::sl1, see above) is sufficient to modify growth, architecture and yield in transgenic Arabidopsis plants. In Examples 5 and 6, some measurements (in particular that of seed yield) made on plants grown as controls (untransformed wild-type plants) yielded values which appeared to be somewhat lower than might normally be expected of wild-type A. thaliana. If this were the case, the net effect would be to exaggerate the differences between the transgenic lines and wild-type plants. In this Example, the results of additional experiments are described wherein the data from measurements made on wild-type control plants were more in line with expectations. The data presented in this Example again shows that constitutive expression of several individual cis-prenyltransferase genes resulted in an enhanced rate of growth to maturity and considerably increased seed yield, characteristics of central importance to the commercial uses of plants.

### Growth Conditions

Seed obtained from lines homozygous for resistance to kanamycin (Rpt1 8-1-5, Spt1 10-4-3, Hpt2 16-2 and Apt5 1-4; Example 4) were sown in Metro-Mix and grown in either constant light at 22° C., with 50% relative humidity, or with a 16 hr photoperiod (fluorescent supplemented with incandescent) at 22° C. (day) and 20° C. (night), with 60% relative humidity. Plants were watered with a nutrient solution containing 1 mM ammonium phosphate, 1 mM potassium nitrate, 1 mM calcium nitrate, 2 mM magnesium sulfate, 1 mM ammonium nitrate, 5 ppb Fe and the following trace elements: manganese, chloride, boric acid, zinc sulfate, cupric sulfate, and molybdic acid.

Plants were observed during growth to determine whether the transgenes, overexpressed using the 35S promoter, affected growth rate or seed yield. Overall, it was observed that most plants constitutively expressing cis-prenyltransferase transgenes exhibited accelerated growth to maturity and higher seed yield as compared to untransformed (wild-type) plants.

### Leaf Growth

An effect of transgene expression on leaf dimensions was observed, with the transgenic plants exhibiting on average longer leaves (Table 12) and broader leaves (Table 13). Leaf length was measured as leaf rosette radius; leaf width was measured at widest point.

TABLE 12
Effect of cis-prenyltransferase transgene expression
on leaf rosette radius (16 h photoperiod)

| Overexpressed | Rosette radius (mm) at | Rosette radius (mm) at 30 |
|---|---|---|
| transgene | 18 dps¹ (std. dev.) | dps¹ (std. dev.) |
| None (wild-type) | 11.72 (4.22) | 34.26 (8.01) |
| Rpt1 | 10.01 (3.91) | 28.63 (8.17) |
| Apt5 | 19.29 (6.93) | 44.47 (5.92) |
| Spt1 | 15.3 (4.52) | 39.11 (6.43) |
| Hpt2 | 16.81 (4.88) | 42.80 (7.42) |

¹Days post sowing

TABLE 13
Effect of cis-prenyltransferase transgene expression
on leaf width (growth with a 16 hr photoperiod)

| Overexpressed | Leaf width (mm) at | Leaf width (mm) at |
|---|---|---|
| transgene | 18 dps¹ (std. dev.) | 30 dps¹ (std. dev.) |
| None (wild type) | 5.99 (1.69) | 10.14 (1.6) |
| Rpt1 | 4.73 (1.54) | 9.17 (3.24) |
| Apt5 | 8.13 (1.82) | 12.62 (1.59) |
| Spt1 | 7.39 (1.76) | 11.44 (1.58) |
| Hpt2 | 7.62 (1.32) | 13.4 (1.66) |

¹Days post sowing

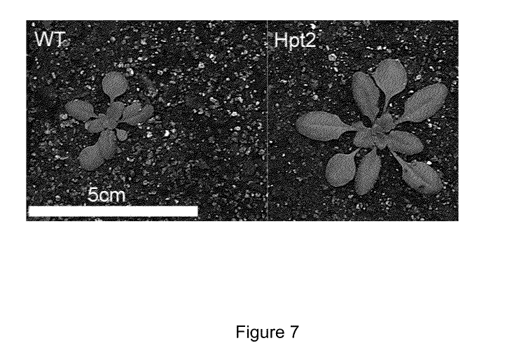

Both of these parameters were increased ca. 1.5-fold in plants expressing the soybean and Hevea cis-prenyltransferases, for example.
FIG. 7 illustrates the differences between a representative wild-type plant and representative transgenic 35S::Hpt2 plant, at 18 days (16 h photoperiod) post sowing.

### Plant Height

Marked effects of transgene expression were also observed on plant height, when compared at fixed times after sowing (Table 14).

TABLE 14
Effect of cis-prenyltransferase transgene expression
on height of bolting plants (16 h photoperiod))

| Overexpressed | % plants bolting at | Height (mm) at |
|---|---|---|
| transgene | 30 dps¹ | 30 dps¹ (std. dev.) |
| None (wild-type) | 50 | 66.49 (59.95) |
| Rpt1 | 61.9 | 40.0 (47.51) |
| Apt5 | 92 | 180.48 (93.68) |
| Spt1 | 100 | 141.06 (81.47) |
| Hpt2 | 96.42 | 106.08 (64.68) |

¹Days post sowing

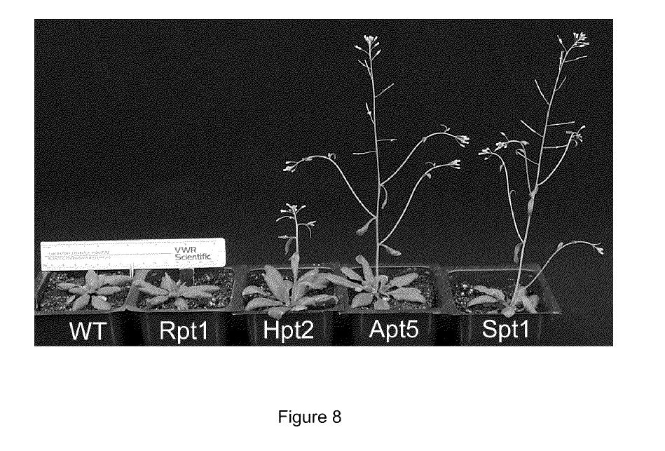

The data shows that bolting plants expressing Apt5, Spt1 or Hpt2 were roughly 2× taller than wild-type plants 30 days after sowing. This effect was not observed in plants expressing Rpt1, which exhibited considerable variability for this measurement and whose average height was essentially indistinguishable from wild-type plants at the time measurements were made. Table 14 also shows that almost all Apt5, Spt1 and Hpt2 transgenic plants were producing bolts at 30 days post sowing, compared to only 50% of wild-type plants. FIG. 8 illustrates the data shown in Tables 12-14, showing that representative plants constitutively expressing cis-prenyltransferases Apt5, Spt 1 and Hpt2 displayed a modified (enhanced) growth rate, as compared to a wild type plant. At 28 dps, any effect of the Rpt1 transgene is not obvious in this illustration.

### Flowering and Seed Yield

The effect of the transgenes on flowering time and seed yield were also monitored (Tables 15, 16). The data shows that plants overexpressing Apt5, Hpt2 and Spt1 on average produced inflorescences earlier than wild-type plants. The most dramatic effect of transgene expression, however, was on final seed yield.

Seed yield was determined according to the average weight of seed per plant, since individual seed weight was not affected by expression of any of the transgenes. More specifically, 1000 seeds from either the wild-type or the transgenics plants (grown under constant light or with a 12 hr photoperiod) always weighed ca. 30 mg.

The data (Table 16) show that plants overexpressing cis-prenyltransferases produce more numerous seed than untransformed, wild-type plants. Those overexpressing Apt5, Hpt2 and Spt1 are capable of yielding 1.5× the number of seed that can be routinely obtained from wild-type Arabidopsis.

TABLE 15
Effect of cis-prenyltransferase transgene expression
on time to flowering (16 h photoperiod))

| Overexpressed | Days to flowering |
|---|---|
| transgene | (std. dev.) |
| None (wild-type) | 27.86 (2.58) |
| Rpt1 | 28.42 (2.33) |
| Apt5 | 23.04 (3.03) |
| Spt1 | 23.61 (3.26) |
| Hpt2 | 24.53 (2.02) |

TABLE 16
Effect of cis-prenyltransferase transgene expression
on seed yield (growth with constant light)

| | Average weight of |
|---|---|
| Overexpressed transgene | seed per plant in mg (std. dev.) |
| None | 132.06 (29.41) |
| (wild-type) | |
| Rpt1 | 45.06 (12.57) |
| Apt5 | 204.08 (63.72) |
| Spt1 | 212.1 (65.03) |
| Hpt2 | 206.26 (68.35) |

In summary, the data clearly shows that over-expression of cis-prenyltransferase genes in Arabidopsis results in dramatic modification to the growth rates and/or yield and/or architecture of the transgenic plants, as compared to the wild-type. With the majority of the exogenous transgenes, seed yields, plant size and leaf area were also increased, which could translate into more biomass per pot (or acre). This could be an especially beneficial result in any plant species that is grown commercially for biomass production (e.g., sugarcase, tobacco, cotton, etc.).

What is claimed is:

1. A method for producing a transformed plant having an altered growth phenotype as compared with an untransformed plant comprising:
a) transforming a plant cell with an isolated nucleic acid molecule encoding a cis-prenyltransferase under the control of suitable regulatory sequences;
b) recovering a transformed plant cell produced in step (a);
c) regenerating a plant from the transformed plant cell of step (b); and
d) growing the transformed plant produced in step (c) under conditions wherein the isolated nucleic acid molecule encoding a cis-prenyltransferase is expressed and the growth phenotype of the transformed plant is altered.

2. A method according to claim 1 wherein the cis-prenyltransferase comprises a domain as defined by the amino acid sequence selected from the group consisting of SEQ ID NOs: 7-10.

3. A method according to claim 1 wherein the cis-prenyltransferase comprises a domain as defined by the amino acid sequence selected from the group consisting of SEQ ID NOs: - 11-13.

4. A method according to claim 1 wherein the cis-prenyltransferase is a polypeptide having the amino acid sequence selected from the group consisting of SEQ ID NO:2, SEQ ID NO:4, SEQ ID NO:6 and SEQ ID NO:16.

5. A method according to claim 1, wherein the isolated nucleic acid molecule encoding a cis-prenyltransferase is isolated from the group consisting of plants and microbes.

6. A method according to claim 1 wherein the plant cell is selected from, but not limited to the group consisting of Hevea brasiliensis, Taraxacum spp., tobacco (Nicotiana spp.), tomato (Lycopersicon spp.), potato (Solanum spp.), hemp (Cannabis spp.), sunflower (Helianthus spp.), sorghum (Sorghum vulgare), wheat (Triticum spp.), maize (Zea mays), rice (Oryza sativa), rye (Secale cereale), oats (Avena spp.), barley (Hordeum vulgare), rapeseed (Brassica spp.), broad bean (Vicia faba), french bean (Phaseolus vulgaris), other bean species (Vigna spp.), lentil (Lens culinaris), soybean (Glycine max), arabidopsis (Arabidopsis thaliana), guayule (Parthenium argentatum), cotton (Gossypium hirsutum), petunia (Petunia hybrida), flax (Linum usitatissimum), carrot (Daucus carota sativa), tea, celery, brussel sprout, artichoke, okra, squash, kale, asparagus (Asparagus), banana (Musa), blueberry (Vaccinium), cacao (Theobroma), capsicum pepper (Capsicum), cassaya (Manihot), cucumber (Cucumis), eggplant (Solanum), lettuce (Lactuca), mango (Mangifera), oilseed rape, canola, cabbage, broccoli, cauliflower (Brassica), onions (Allium), papaya (Canca), peas (Pisum), peanut (Arachis), pineapple (Ananas), pinto bean, mung bean, pumpkin, zucchini (Cucurbita), radish (Raphanus), sesame (Sesame), spinach (Spinaceae), sorphum (Sorphum), strawberry (Fragana), sugarcane (Saccharum), sugar beet (Beta), sweet potato (Ipomoea), watermelon (Citrullus), yam (Dioscorea), alfalfa (Medicago), amaranth (Amaranthus), angelica (Agelica), castorbean (Ricinus), colewort (Crambe), jojoba (Simmondsia), jute (Corchorus), kenaf (Hibiscus), lupine (Lupinus), plantain (Plantago), sisal (Agave), snapdragon (Antirrhinum), switch grass (Panicum), apple (Malus), acacia (Acacia), chestnut (Castanea), citrus (Citrus), coconut (Cocos), coffee (Coffea), cypress (Cupressus), eucalypti (Eucalyptus), grape (Vitis), hemlock (Tsuga), hickory (Carya), maple (Acer), oak (Quercus), pear (Pyrus), peach, plum, cherry (Prunus), pine (Pinus), poplar (Populus), rose (Rosa), spruce (Picea), and walnut (Juglans).

7. A method according to claim 1 wherein the suitable regulatory sequences comprise a promoter sequence.

8. A method according to claim 7, wherein the promoter sequence is selected from the group consisting of:
a) constitutive plant promoters;
b) plant tissue-specific promoters;
c) plant development-stage-specific promoters;
d) inducible promoters; and
e) viral promoters.

9. A method according to claim 8, wherein the tissue-specific promoters are selected from the group consisting of:
a) male germline promoters;
b) female germline promoters;
c) common germline promoters;
d) flower promoters;
e) vegetative shoot apical meristem promoters;
f) floral shoot apical meristem promoters;
g) stem promoters;
h) meristem promoters;
i) tuber promoters;
j) seed promoters;
k) endosperm promoters;
l) root promoters;
m) nodule promoters;
n) embryo promoters;
o) leaf promoters; and
p) fruit promoters.

10. A method according to claim 1 wherein the altered growth phenotype is increased growth rate of the transformed plant.

11. A method according to claim 10 wherein the increased growth rate is defined by characteristics selected from group consisting of:
decreased time to germination, increased root growth rate, increased shoot growth rate, decreased time to flowering, decreased time for fruit maturation, and decreased time of seed setting.

12. A method according to claim 1 wherein the altered growth phenotype is increased yield of the transformed plant.

13. A method according to claim 12 wherein the increased yield is defined by characteristics selected from group consisting of: increased total biomass, increased root growth, increased shoot growth, increased seed set, increased seed production, increased grain yield, increased fruit size, increased nitrogen fixing capacity, increased nodule size, increased tuber formation, increased stem thickness, increased endosperm size, and an increased number of fruit per plant.

14. A method according to claim 1 wherein the altered growth phenotype is a modified plant architectural trait.

15. A method according to claim 14 wherein the modified architectural trait is selected from the group consisting of: modifications in the shape, size, number, color, texture, arrangement and patterning of the root, leaf, shoot, fruit, petiole, trichome, flower, sepals, petal, hypocotyl, stigma, style, stamen, pollen, ovule, seed, embryo, endosperm, seed coat, aleurone, fibre nodule, cambium, wood, heartwood, parenchyma, sclerenchyma, seive element, phloem, or vascular tissue.

16. A method for altering the growth phenotype of a plant as compared with an untransformed plant comprising:
a) providing a plant comprising a gene encoding a cis-prenyltransferase; and
b) upregulating the gene of (a) wherein the growth phenotype of the plant is altered.

17. A method according to claim 16 wherein the gene encoding a cis-prenyltransferase is endogenous to the plant.

18. A method according to claim 16 wherein the gene encoding a cis-prenyltransferase is exogeneous to the plant.

19. A method according to claim 16 wherein the gene encoding a cis-prenyltransferase is under the control of an inducible promoter.

20. A method according to claim 16 wherein the gene encoding a cis-prenyltransferase is expressed on a multicopy plasmid.

21. A plant produced by the method of claim 1.

22. A plant expressing a foreign cis-prenyltransferase gene having an altered growth phenotype.

23. A plant having a growth phenotype altered by the method of claim 16.

24. A plant expressing a foreign cis-prenyltransferase gene having an altered yield phenotype.

25. A plant having a yield phenotype altered by the method of claim 16.
